(19) United States
(12) Patent Application Publication
(10) Pub. No.: US 2003/0138774 A1
(43) Pub. Date: Jul. 24, 2003

(54) Methods and apparatus for separating and detecting nucleic acid

(76) Inventors: Chris L. Jones, Riverdale, NJ (US); Ezra S. Abrams, Newton, MA (US); Stephen G. Kieffer-Higgins, Dorchester, MA (US); Tianhong Zhang, Ashland, MA (US); Graham P. Eacock, Westborough, MA (US); Michael Webb, Dunstable, MA (US); Christopher S. McDowell, Maynard, MA (US)

Correspondence Address: Hollie L. Baker, Hale and Dorr LLP, 60 State Street, Boston, MA, 02109, US

(21) Appl. No.: 09/766,880

(22) Filed: Jan. 19, 2001

Related U.S. Application Data
(60) Provisional application No. 60/176,839, filed on Jan. 19, 2000.

(51) Int. Cl. C12Q 1/68; G01N 33/53

(52) U.S. Cl. 435/6; 435/7.1; 204/461

(57) ABSTRACT

A thin gel for separating and detecting a target molecule, (e.g., an analyte) and methods of use thereof is described. The gel can be optionally reinforced to facilitate removal from an electrophoretic apparatus, wherein the gel matrix comprises immobilized (e.g., covalently bound) capture probes capable of specifically hybridizing with, or binding to, the analyte to be detected. The gel is thin and very short assay times can be achieved facilitating rapid detection of analytes. An assay apparatus is suitable for use with an electrophoretic assay system for the detection of, or separation and detection of an analyte in a biological sample is also described.

### RELATED APPLICATION(S)

This application claims the benefit of U.S. Provisional Application No. 60/176,839 filed Jan. 19, 2000, the entire teachings of which are incorporated herein by reference.

### BACKGROUND OF THE INVENTION

Separation and detection of nucleic acid molecules and fragments is desirable for diagnostic, analytical, and research purposes. Nucleic acids include DNA, RNA, and analogues thereof. Different species of microbes such as bacteria, viruses, fungi, and/or parasites have unique genetic (DNA and/or RNA) sequences that can be used to detect the presence or absence of a particular microbe. Common sequences of nucleic acids are also recognized within a group of microbes such as in bacteria, which can be used to detect the presence or absence of members of that particular group. Further, when DNA or RNA sequences are modified, such as by single nucleotide polymorphisms (SNPs) or mutation, disease or dysfunction can result. Detection of polymorphisms or mutations can facilitate early intervention during the life of a patient in the form of medication or education to improve quality of life. For example, when genetic analysis indicates that a child will be born with cystic fibrous, early medical intervention can be provided that can minimize complications of the disease.

A biological sample can be composed of a heterogeneous mixture of components, for example, proteins and nucleic acids. Especially for diagnostic applications, the ability to rapidly separate the components and identify a target molecule, e.g., a particular DNA, RNA or protein, in a sample can be a limiting factor in developing a useful diagnostic test. For example, when a patient requires an infusion of platelets, knowledge is needed of whether or not the platelets are contaminated with bacteria at the moment of transfusion. During the time required to complete the test, the bacteria continue to multiply. Thus, as more time elapses the usefulness of the platelets may change.

Electrophoresis on a gel is one method traditionally used for separating complex samples containing nucleic acid molecules and fragments into individual bands having unique sequences. Both the weight and the charge of the molecule or fragment can affect its speed of movement through the electrophoretic medium. For nucleic acids which have a uniform charge to mass ratio, the larger the nucleic acid the more slowly it moves within the gel. Also the distance that the sample must travel before it is resolved into its component parts effects the speed of separation using this method. Traditional electrophoretic methods of analyzing nucleic acids typically requires highly trained technicians and, depending on the type of sample and equipment used, takes from 2-48 hours to complete.

It is apparent that methods and apparatuses to rapidly and reliably separate and detect target molecules in biological samples are needed.

### SUMMARY OF THE INVENTION

The present invention relates to a system for detecting, separating, purifying and/or concentrating a component or target molecule, e.g., an analyte, in a biological sample by allowing an electric field to transport the component into the vicinity of a capture probe, wherein the component specifically binds to or hybridizes with a capture probe. The system can include a housing, e.g., an electrophoresis cassette for receiving at least two electrodes; one or more structures for receiving a thin gel matrix that includes a capture probe; a source of ions to support an electric field, e.g., a buffered salt solution. The capture probe is, optionally contained within a gel matrix, for example agarose. Optionally, the apparatus further includes structures for holding the thin gel or for holding a reinforced thin gel; structures for introducing a sample into a sample compartment in the system, wherein the sample potentially contains a component that binds to, or hybridizes with, the capture probe, e.g., a nucleic acid sequence which is substantially complementary to the capture probe. The system can further include an electrical power source, or can include structures for connecting the system, or for connecting a part of portion of the system to an electrical power source.

The system of the present invention can be used for determining the presence or absence of a component or target molecule. For example, a sample can be transferred to the electrophoresis cassette, e.g., the sample is introduced into a sample compartment of the cassette and an electrical force, e.g., a voltage gradient is applied across the electrodes of the cassette resulting in one or more components of the sample to pass from the sample compartment through the thin gel matrix. One or more capture probes are associated with the gel. As used herein, a capture probe may be associated with a gel by affixing, immobilizing, entrapping, and/or by chemically, ionically, or covalently binding the capture probe to the gel. The capture probe can also be referred to herein as a ligand. For example, if the target molecule, e.g., the analyte to be detected is a nucleic acid having a nucleotide sequence that is substantially complementary to the nucleotide sequence of the capture probe, the nucleic acid is retained in the thin gel, while nucleic acids lacking a complementary sequences move through the gel. Where the system of the invention is used in a method of detecting, assaying, or analyzing, binding of the target molecule to the capture probe can be detected by one or more chemical or physical properties exhibited by the capture probe-target complex, which property will be known to those skilled in the art. In one embodiment, the capture probe-target complex includes a detectable label.

In one embodiment, a system for capturing an analyte includes an electrophoresis cassette. The electrophoresis cassette has a housing, e.g., a base having a pair of electrode channels, a barrier interposed between the electrode channels, a barrier having at least one migration channel extending between the electrode channels, and a pair of enlarged slots bounded and opening into the migration channel. A first electrode extends in a first electrode channel and a second electrode extends in a second electrode channel. A capture gel holder is receivable in an enlarged slot. The capture gel holder can have an opening aligned with a migration channel. A thin gel is positioned in an opening of a capture gel holder. In one embodiment, the capture gel holder is a comb. The thin gel has a matrix which can be a polymeric mesh; optionally the matrix may be non-conductive.

In one embodiment a cassette includes an evaporation cover positioned to overlie the base of the cassette. The evaporation cover can have at least one opening for a capture gel holder, at least one opening for venting of gas, and/or at least one opening to allow a sample well-forming comb to pass into the migration channel. The cassette can include a pair of wash well sets for receiving the teeth of the capture gel holder. The electrodes can have a pair of terminals extending through the evaporation cover. The terminals can be flush with the top of the evaporation cover.

In one embodiment, a capture gel holder has a handle and a plurality of teeth projecting from the handle. One or more of the teeth can have a bore through the tooth. A polymeric material, e.g., a non-conductive material, can occupy space within the bore of the tooth, so as to be stretch over or within the bore of that tooth. The material can support a gel, e.g., a gel matrix. The bore can be bordered by, preferably surrounded by, a recessed central region, and can further be bordered by, or surrounded by, a flange on the bore so that the recessed central region and the flange facilitate release of gas. At least one tooth has a shape that is adapted to fit in the cassette in a particular or specified direction, for example a keyed shape.

In one embodiment, a thin gel is used for separating and detecting a target molecule, (e.g., an analyte), for example, a nucleic acid sequence, a nucleic acid analogue, where such analogue is for example, a peptide nucleic acid (PNA), a modified nucleic acid sequence, or a polypeptide having a particular binding affinity, a particular structure, or a particular amino acid sequence, in a sample. More particularly, the present invention provides a polymeric gel matrix, optionally reinforced to facilitate removal from an apparatus, e.g., an electrophoretic cassette, wherein the polymeric gel matrix includes capture probes capable of specifically hybridizing with, or binding to, a target molecule. As described herein, the gel is “thin”. The term “thin” as used herein refers to the thick mass of the get matrix. The gel matrix of the present invention has the attributes of either a filter or a sieve, so as to retain one or more sample components on the gel matrix while permitting other components to pass through. It is further desirable that the gel matrix of the present invention be capable of manipulation, so that it can be placed into and removed from e.g., the electrophoretic cassette. It can be appreciated by one of skill in the art, that by reducing the thickness of the gel matrix greater assay speeds may be attained. However, the term “thin” can also refer to characteristics of selective permeability and ease of handling so as to decrease the amount of time required to achieve desired detection, separation, or concentration. Importantly, using the methods and apparatuses described herein, no prior amplification of sample analytes is required, yet the sensitivity of this rapid assay is comparable to more complex and time consuming methods of analysis.

The gel can comprise any material that can be used in an electrophoretic assay system for detecting, separation, or concentrating an analyte, such as a form of polyacrylamide, agarose, starch, or dextran. The gel may be formulated so that it is stable enough to be relocated, moved, or physically transported by manual or mechanical means. The gel matrix can be provided with reinforcement that acts to reinforce the gel thereby facilitating handling. When the thin gel matrix is cast on a reinforcing material, the thin gel matrix is called a reinforced thin gel. In one embodiment, the thin gel reinforcement includes a non-conductive porous material such as a mesh, a web, a mat, or a felt. In a second embodiment, the thin gel includes a polymeric additive, cross-linking agent, or other chemical agent that can be incorporated into the gel so as to structurally reinforce the gel matrix. The reinforcement increases the strength and durability of the gel matrix thereby rendering it resistant to tearing or stretching. Furthermore, the gel can be used with a holder that facilitates transfer and processing of the gel within a detector or detection station to detect binding of the target molecule to a cassette probe contained in the gel.

In another embodiment, a highly sensitive, non-amplified, rapid assay for the detection of a target molecule in a biological sample is provided. A sample, e.g., sample suspected to be contaminated with an undesirable species, e.g., a target molecule such as a microbial species, e.g., bacteria, fungus, or virus, can be processed as described herein to render a nucleic acid characterized as present in that species, preferably nucleic acid known to be unique to that species, available to hybridize to a capture probe associated with the gel matrix on the capture gel holder. The hybridized nucleic acid is thereby captured on the gel, and can then be detected and reported.

In this embodiment of the present invention, a biological sample is first diluted with an appropriate diluent, and then centrifuged at high speed to pellet any target bacteria present in the sample. The pelleted bacteria is treated in a manner to solubilize membrane components, and render target bacterial nucleic acids suitable for hybridization with specific nucleic acid probes. Buffer containing alkaline phosphatase-conjugated reporter probes is added to the sample and the sample is heated to temperatures sufficient for unfolding of target nucleic acids and hybridization of reporter probes. The samples are cooled, and an aliquot of each sample is loaded into a pre-warmed electrophoresis cassette as described herein, and a voltage is applied. The samples migrate past capture combs (also referred to herein as capture gel holder) containing capture probes specific to the target bacterial nucleic acids. The capture probes are immobilized on a thin gel. The combs are then dip-washed and moved to a slot in the cassette which is upstream of the original electrophoretic path. A voltage is again applied for a brief time to electrophoretically wash the combs. The combs are placed in a conditioning buffer for a set time, then removed and are placed flat in a reader tray. Enzyme substrate is added in a quantity sufficient to react with the reporter probes hybridized to target immobilized in the gel. The reader trays are then scanned by the reader, and any signal present is detected and reported. The detection of signal is indicative of the presence of the target bacteria in the sample.

In another embodiment, for example, a sample suspected of bacterial contamination can be cultured to grow up any colonies of bacteria present in the sample and the culture is transferred to the thin gel matrix by blotting the sample with an agarose gel encapsulated polyester or nylon membrane. The transferred bacterial colonies can be exposed to conditions that result in lysis of the bacteria and release of the analyte or analytes, e.g., nucleic acids contained therein. The nylon membrane can then be sandwiched against a thin gel matrix comprising capture probes, specific for the bacteria to be detected and any air bubbles between the two are eliminated, e.g., by gentle pressure. The sandwich can then be placed in the electrophoretic apparatus and, under suitable conditions, an electrical current is applied and the bacterial components comprising the analyte or analytes to be detected migrate (i.e., move) into contact with the immobilized capture probes. If the bacterial analyte to be detected is present within the sample, the analyte can hybridize, or bind to, the immobilized probe and remain immobilized (i.e., does not further migrate) in the gel. Bound analyte can then be detected, e.g., by hybridization of the analyte to a reporter probe and reported as described herein.

In addition, the methods and apparatuses described herein permit rapid capture and purification of hybridized nucleic acids. For example, using the methods and apparatus described herein, target nucleic acids can be rapidly isolated from a complex sample and specifically captured by the immobilized capture probes of the gel on the capture comb. Once the target nucleic acid is captured and the remaining contaminants from the sample are removed from the presence of the target, the capture comb can be removed to a different set of slots in the same electrophoretic cassette, a separate container, or another apparatus where the immobilized target/probe complex is subjected to conditions which release the target from the probe, thus the target molecule is recovered from the sample and can be used in further procedures such as for polymerase chain reaction.

In addition, confirmatory testing for the presence of a specific polymerase chain reaction (PCR) product can be performed using the methods and apparatus of the present invention. A complex PCR reaction mixture can be contacted with immobilized capture probes on a capture comb and the specific PCR product can be captured and detected to confirm the accurate performance of the PCR reaction.

As a result of the invention described herein, methods and apparatuses are available for the rapid assay and purification of target molecules from complex biological samples. The invention described herein eliminates the necessity for complex and time-consuming chemical reactions, and significantly reduces, or eliminates sample background interference and contamination from environmental nucleic acids that may be detrimental to accurate diagnostic and purification results. For example, using the methods and apparatuses as described herein, a broad panel of clinically-relevant bacterial contaminations at a level of approximately 10⁴ to 10⁵ CFU/ml can be detected, without the need for prior PCR amplification of the sample's nucleic acids. Thus a rapid assay format for detecting bacterial contamination of biological products (e.g., bacterial contamination of blood products such as platelets) with simplified sample processing is now available.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a top perspective view of an electrophoresis cassette;

FIG. 2 is a top perspective view of the electrophoresis cassette of FIG. 1 with the cover;

FIG. 3 is a top view of the electrophoresis cassette;

FIG. 4A is a top perspective view of the evaporation cover;

FIG. 4B is a top view of the evaporation cover;

FIG. 5 is a front perspective view of a capture gel holder;

FIG. 6A is a front view of the capture gel holder;

FIG. 6B is a side view of the capture gel holder;

FIG. 7A is an enlarged front view of a portion of the capture gel holder of FIG. 6A;

FIG. 7B is an enlarged front view of a portion of the capture gel holder of FIG. 6A;

FIG. 7C is a sectional view taken along the line 7C-7C in FIG. 6A;

FIG. 7D is a sectional view taken along the line 7D-7D in FIG. 6A;

FIG. 7E is a sectional view taken along the line 7E-7E in FIG. 6A;

FIG. 7F is a sectional view taken along the line 7F-7F in FIG. 6A;

FIG. 7G is a sectional view taken along the line 7G-7G in FIG. 6A;

FIG. 8A is a top perspective of a sample well-forming comb for making sample wells in the electrophoresis cassette;

FIG. 8B is a top perspective view of a molding comb;

FIG. 8C is a top perspective view of an alternative molding comb;

FIG. 9A is a schematic of an exemplary method of determining if bacteria is present in a biological sample;

FIG. 9B is a second schematic of an exemplary method of determining if bacteria is present in a biological sample;

FIG. 9C shows SEQ ID Nos. 1-17, RNA sequences from a panel of target organisms along with the complements of the probe sequences. The lines enclosed by the thin boxes indicates the DNA sequence that is complementary to the probes. The white dotted lines underline the sequences corresponding to the capture probes that are linked to the thin gel membrane. The solid white lines underline the sequences corresponding to the reporter probes.

FIG. 10 is a schematic of a method for rapid assay of an analyte in a sample;

FIG. 11A illustrates a front perspective view of a capture gel reinforced with a non-conductive mesh;

FIG. 11B is a cross-sectional view of the capture gel taken along the line 11B-11B of FIG. 11A;

FIG. 12A illustrates a side view of an alternative embodiment of the capture gel having an encapsulating enmeshed glass fibers;

FIG. 12B is a cross-sectional view of the capture gel taken along the line 12B-12D of FIG. 12A;

FIG. 13 is a side perspective view of an electrophoresis machine with a side removed;

FIG. 14 is a side perspective view of the electrophoresis machine and overlying incubator unit;

FIG. 15A is a top view of a tray for receiving a pair of capture gel holders for use in a detection device such as a luminescence reader;

FIG. 15B is a sectional view of the tray taken along the line 15B-15B in FIG. 15A;

FIG. 16 is a schematic representation of an embodiment of the electrophoretic cassette and a detection station in which a target detection is accomplished by an enzyme stimulated chemiluminescence reaction;

FIG. 17 is a schematic outline of a method of purification;

FIG. 18A is a front view of a capture gel holder;

FIG. 18B is a perspective view of the capture gel holder of FIG. 18A;

FIG. 19A is a top view of an alternative embodiment of an electrophoresis cassette;

FIG. 19B is a sectional view of the electrophoresis cassette taken along the line 19B-19B of FIG. 19A;

FIG. 19C is a side perspective view of the electrophoresis cassette;

FIG. 20 is a side perspective of a comb for making sample wells in the electrophoresis cassettes.

FIG. 21A illustrates in a top view of an alternative embodiment of the electrophoretic parallel channel device;

FIG. 21B is a cross-sectional view of the electrophoresis cassette of FIG. 21B;

FIG. 21C illustrative a gel capture holder having thin gel or reinforced thin gel;

FIG. 22 illustrates a graph of results obtained using the assay method disclosed;

FIG. 23 is a graph showing a dose response for in vitro target transcripts spiked into the assay after the centrifugation step;

FIG. 24 is a graph showing a similar dose response of FIG. 23, but using E. coli spiked into sterile platelet concentrates; and

FIG. 25 depicts the time estimates for running up to 8 samples in the assay method described herein.

### DETAILED DESCRIPTION OF THE INVENTION

The present invention relates to the discovery that target molecule detection can be rapidly achieved by utilizing a thin gel matrix comprising immobilized capture probes. Capture and subsequent detection of the target molecule depends upon the density of the capture probe in the gel rather than upon the distance traveled by the target molecule through the thin gel matrix. Further, incorporation of a reinforcement into the thin gel matrix facilitates its handling and its removal from the device, thereby permitting improved procedures for removing background signals and detection. The reinforcement can be provided by a separate material such as a non-conductive mesh or fibrous mat. Alternatively, reinforcement can be a molecular reinforcement for example a cross-linking molecule that is incorporated into the thin gel matrix that strengthens and/or reinforces the matrix.

In one particular embodiment, the present invention comprises a thin gel, an electrophoretic system for use with the thin gel, and a method of using the electrophoretic system with the thin gel. More specifically, the present invention comprises a reinforced thin gel, an electrophoretic apparatus or device, a sample comb or capture gel holder for use with the electrophoretic device, and a method of using the electrophoretic device and the capture gel holder (sample comb) for separating and subsequently detecting an analyte from a sample.

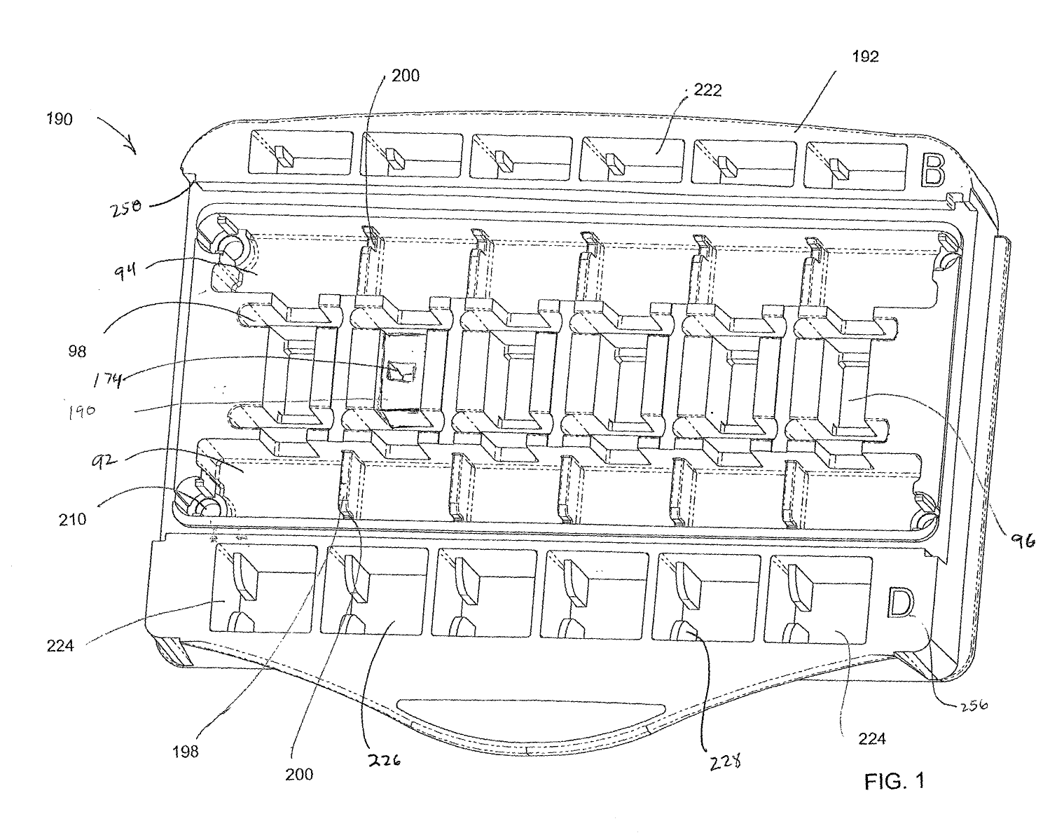

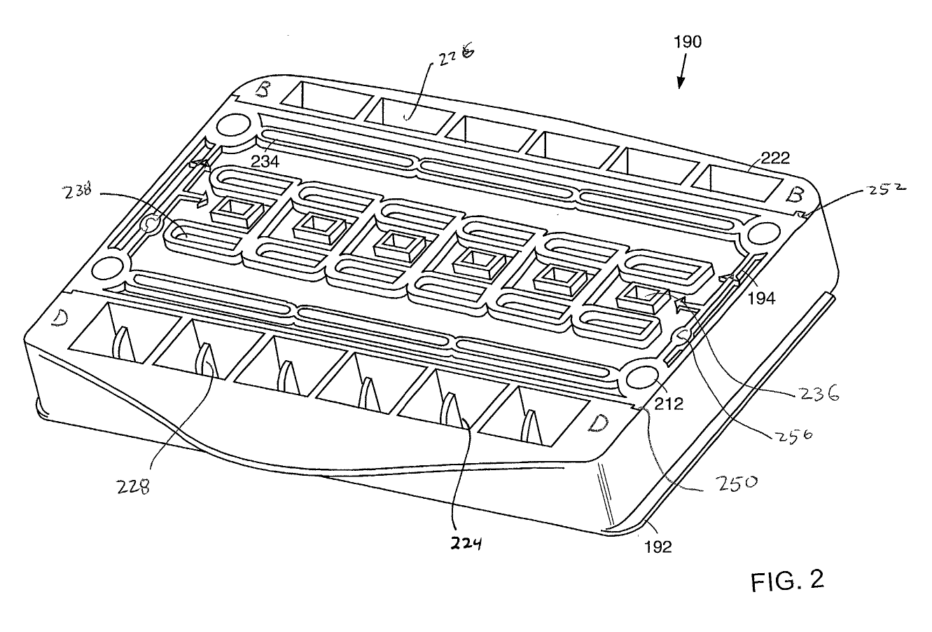

An electrophoresis cassette 190 according to the invention is shown in FIGS. 1 and 2. The cassette 190 has a base 192 and an evaporative cover 194, shown in FIG. 2.

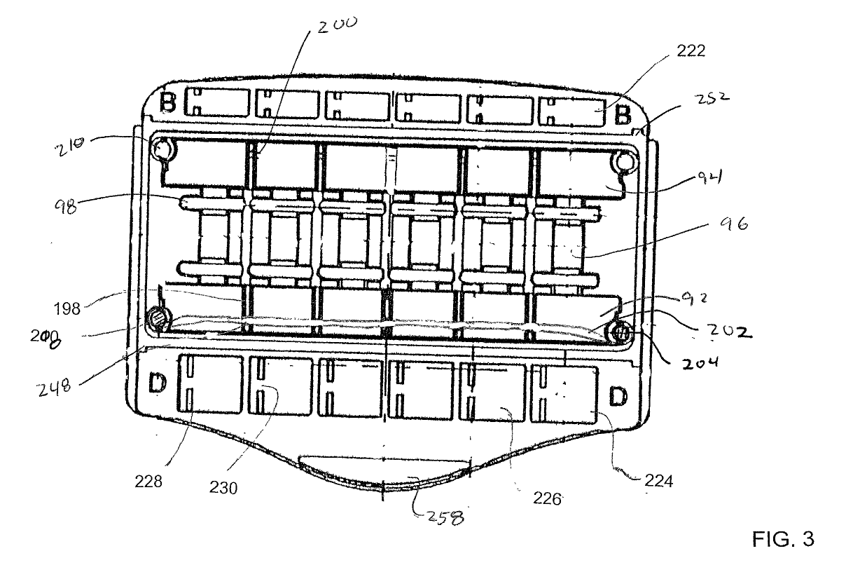

Referring to FIGS. 1 and 3, the base 192 of the electrophoresis cassette 190 has a pair of electrode channels 92 and 94. In each electrode channel 92 and 94 there are a plurality of ribs 198 that break each of the channels into portions. Each rib 198 has a slot 200 through which an electrode 202, such as seen in FIG. 3, extends. The slot 200 of the rib 198 is used to ensure the electrode 202 is properly positioned as explained in detail below. The electrode 202 extends the length of the respective electrode channel 92 and 94. In one embodiment, the electrodes 202 each include a pair of posts 204 and a stainless steel or platinum strip 206 extends between the posts 204 as seen in FIG. 3. A portion of the post 204 is shown for illustrative purposes in FIG. 3, in that the post 204 is not installed in the base 192 until after the evaporative cover 194 is installed. Each post 204 has a cylindrical rod 208, shown in section in FIG. 3, which is received in a bore 210 in the base 192 and an integral circular disk or head 212 which sits on top of the evaporative cover 194, as seen in FIG. 2. The posts 204 are formed of stainless steel in one embodiment.

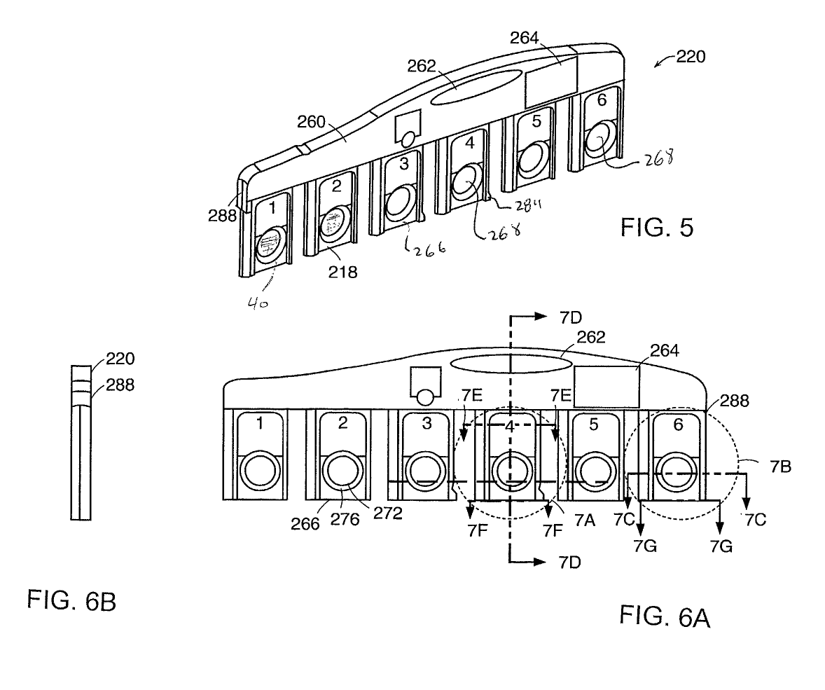

The base 192 has a plurality of channels, the migration channel 96 as seen in FIG. 1, which extend between the electrode channels 92 and 94. Within each migration channel 96 there is a pair of enlarged slots 98 adapted to receive a tooth 218 of a capture gel holder 220. The capture gel holder 220 is further described below with reference FIGS. 5-7G. An alternative embodiment of the capture gel holder 66 is described briefly below. Whether a capture gel holder 66 can be used with a particular electrophoresis cassette 190 is dependent on numerous factors including spacing and dimension of teeth and any keying features.

Referring to FIGS. 1-3, the base 192 of the electrophoresis cassette 190 has a pair of sets of wash wells 222 and 224 FIG. 1. Each set of wash wells 222 and 224 has a wash well 226 associated with and spaced similarly to the migration channel 96. Each wash well 226 is adapted to receive one of the teeth 218 of the capture gel holder. Each of the wash wells 226 of each set of wash wells 222 and 224 has a rib or support 228 with a slot 230 for receiving a tooth 70 or 218 of the capture gel holder 60 or 220. The rib 228 with the slot 230 is offset so that it does not align with the migration channel 96 therein holding the capture gel holder 220 aligned and upright while not interfering with the capture gel 40, as seen in FIG. 5, ensuring that the bore 76 in each tooth 218 which holds the capture gel 40 is accessible by the solution in the wash well.

In one embodiment, the wash wells 226 of the set of wash wells 224 have a larger capacity or volume than those of the other set of wash wells 222. The purpose of the difference in size is to allow the capture gel 40 to react with different amounts and concentrations of solutions or reagents. For example, in one embodiment, the set of wash wells 224 is referred to as a set of conditioner wells and the other set of wash wells 222 is referred to as the set of wash wells.

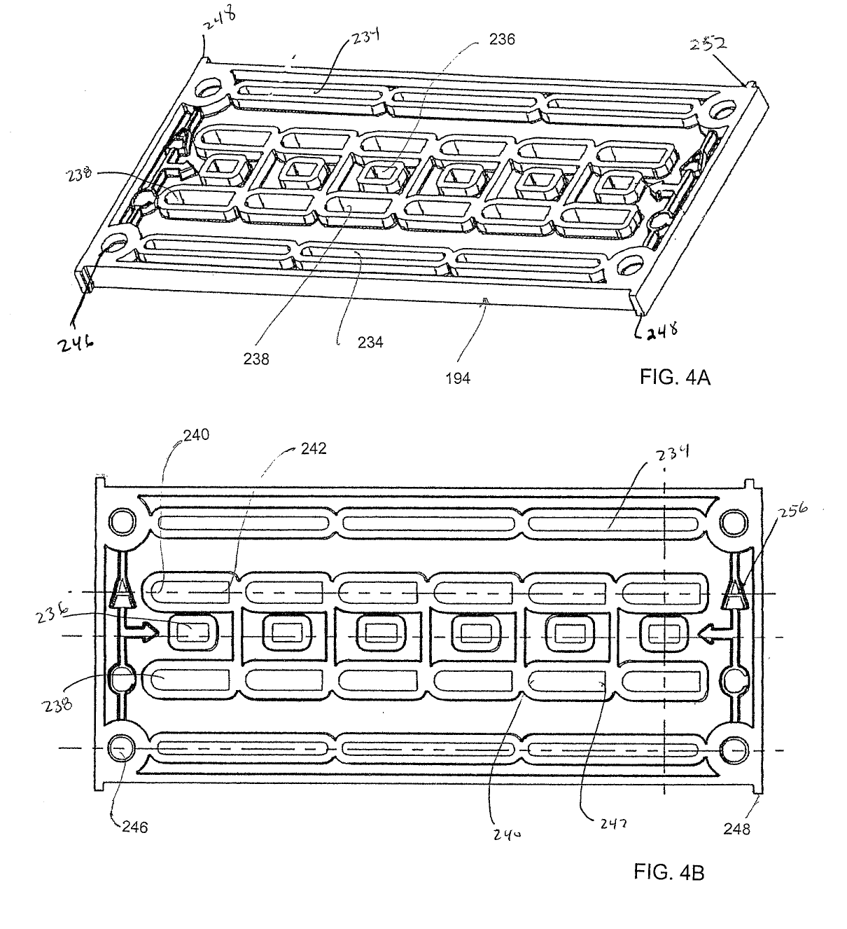

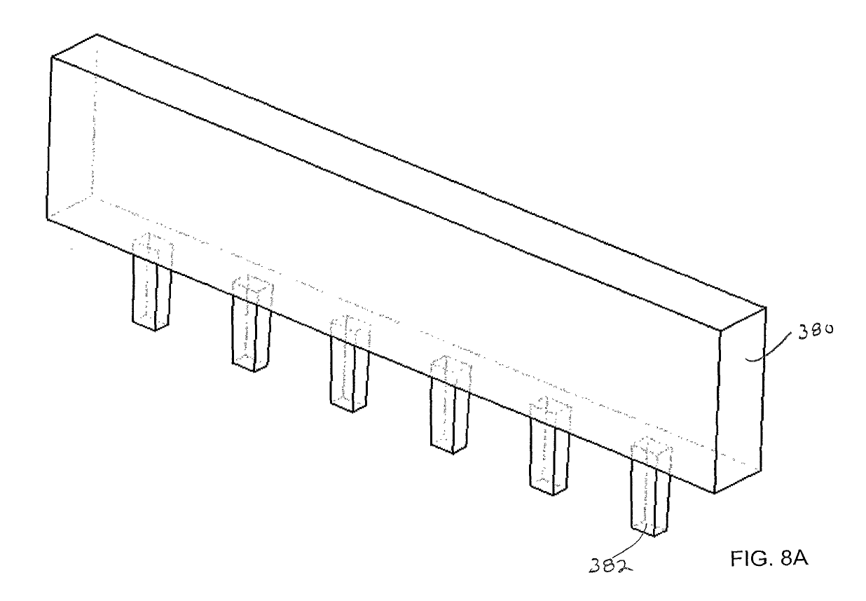

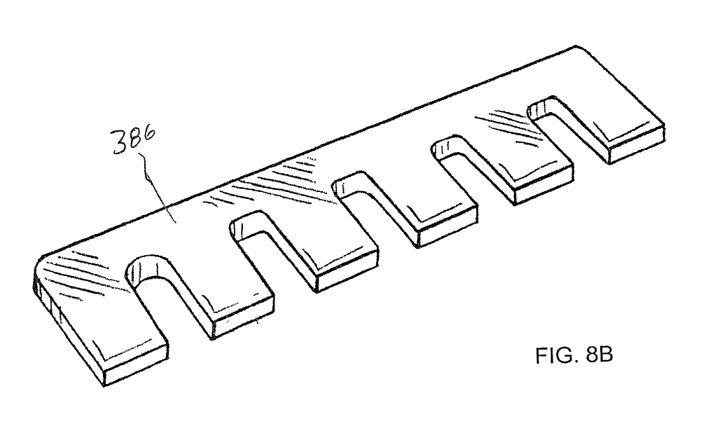

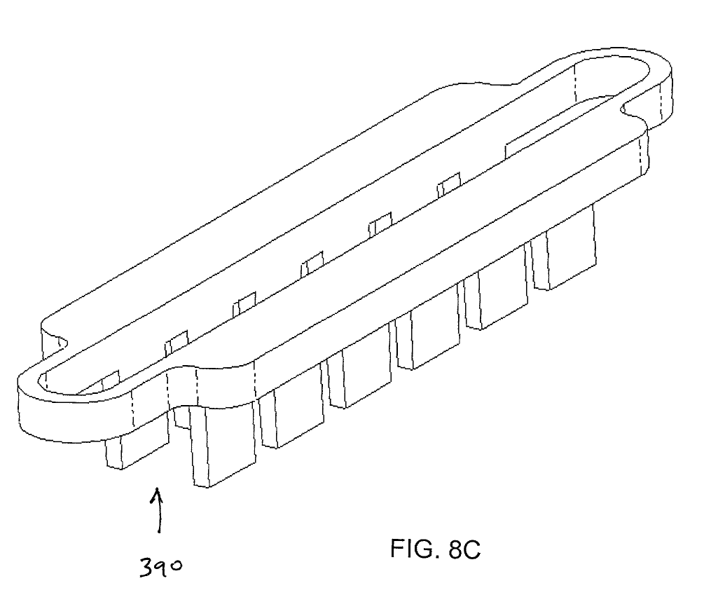

Referring to FIGS. 2, 4A, and 4B, the evaporative cover 194 has a plurality of vent holes or slots 234 for overlying the electrodes 202. The evaporative cover 194 in addition has a plurality of generally square openings 236. The square openings 236 allow a plurality of teeth 382, as seen in FIG. 8A, of a sample well-forming comb 380, to pass into the migration channel 96.

In addition, still referring to FIGS. 2, 4A, and 4B, the evaporative cover 194 has a plurality of slots 238 for receiving the teeth 218 of the capture gel holder 220 and which overlies the enlarged slot 98 of the migration channel 96, as seen in FIG. 1, of the base 192. In one embodiment, the slots 238 have a rounded side edge 240 and a squared side edge 242 as best seen in FIG. 4B. The slots 238 are shaped so that the capture gel holder 220 can only pass through the slots 238 in a certain orientation, as explained in further detail below with reference FIGS. 5-7G. In one embodiment, the evaporation cover 194 is attached to the base 192 by ultrasonic welding.

The evaporative cover 194 does not cover the sets of wash wells 222 and 224. As indicated above, the circular disk 212 of the post 204 at the electrode 202 overlies the evaporative cover 194. The evaporative cover 194 has a hole 246 through which the cylindrical rod of the post 204 extends.

Still referring to FIGS. 4A and 4B, the evaporative cover 194 has a plurality of tabs 248 to be received in complimentary notches 250 in the base 192 of the electrophoresis cassette 190, as seen in FIGS. 1 and 2. One of these tabs, tab 252, of the tabs 248 is positioned slightly inward of the position of the other tabs 248 as seen in FIGS. 4A and 4B such that the evaporative cover 194 can only be placed on the base 192 in one direction. In one embodiment, both the evaporative cover 194 and the base 192 have markings 256 such as the letters “A,” “B,” “C,” and “D” adjacent to the slots for the teeth 238 and the sets of washing wells 222 and 224 to indicate the order of receiving (i.e., the steps) the teeth 218 of the capture gel holder 220 that are inserted into the respective components. The steps are described in greater detail below.

Referring back to FIGS. 2 and 3, the electrophoresis cassette 190 has a handle portion 258 on the base 192 for assisting in the movement of the electrophoresis cassette 190.

The electrophoresis cassette 190 is used in conjunction with the capture gel holder in the method of capture of target molecule. The capture gel holder 220 as illustrated in FIG. 5 has a plurality of teeth 218 and a handle portion 260. The handle portion 260 has a recess 262 to assist in grasping and a second recess or label receiving portion 264.

Referring to FIGS. 5 and 6A, each of the teeth 218 has a thin central region 266 in which is located a bore 268 which extends through the tooth 218. The thinning of the central region 266 helps to prevent entrapment of bubbles on the face of the capture gel 40 and tooth 218 when inserting the capture gel holder into the slot 98 and 238 of the assembled and filled cassette.

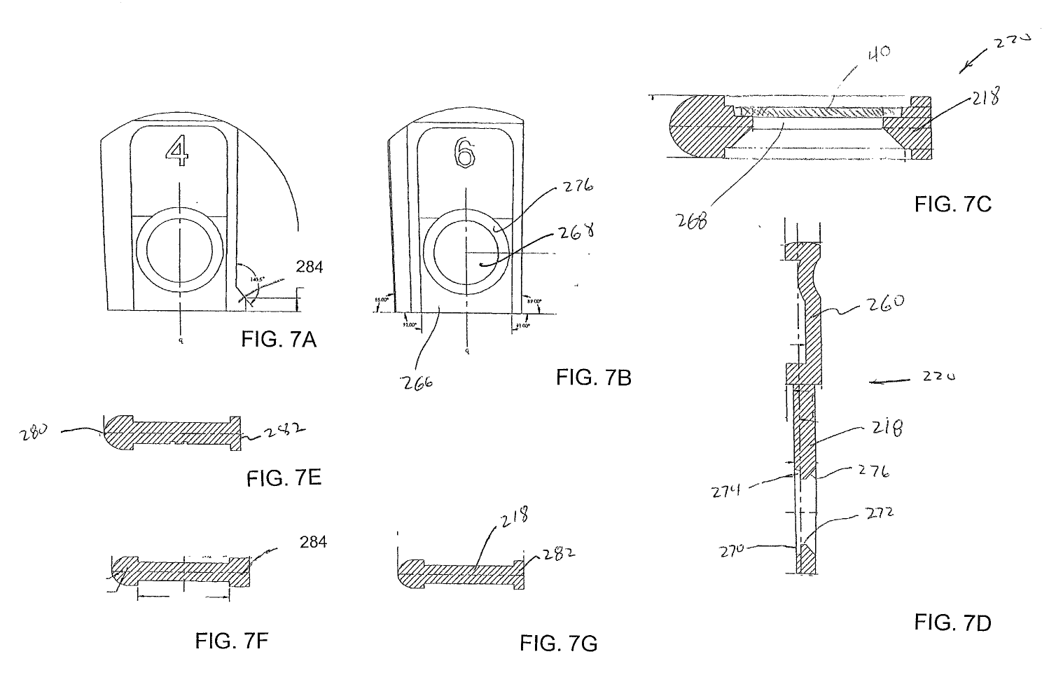

The bore 268 has a larger opening 270 and a smaller opening 272 creating a shoulder 274 as best seen in FIG. 7D. The smaller opening 272 has a taper 276. A capture gel 40 covers or overlies the bore 268 for capture of the target molecules as explained below. As best seen in FIGS. 7B and 7D, the teeth 218 are tapered so that each of the teeth is both narrower and shorter at the bottom as compared to the top of the tooth near the handle.

In one embodiment, the capture gel holder 220 has a length of slightly larger than 4 inches and has six teeth 218. The center lines of adjacent bores 268 are 0.709 inches apart with the spaces between center lines of the first and last bores 268 being 3.543 inches. The teeth 218 are tapered with the bottom having a length of 0.520 inches and a width in the thin central region 266 of 0.08 inches and the top of the tooth, near the handle portion, having a length of 0.545 inches and width in the central region of 0.138 inches.

The bore 268, in one embodiment, has a diameter of 0.260 inches. The top of the bore 268 being 0.381 inches above the bottom of the tooth.

In one embodiment, migration channel 96 has a width of 0.295 inches which is larger than to the diameter of the bore 268 in the tooth 238.

As indicated above with respect to the evaporative cover 194 in FIGS. 4A and 4B where the slots for the teeth 238 had a rounded side 240 and square side 242, the teeth 218 of the capture gel holder 220 have a rounded side edge 280 and a square side edge 282 as best seen in FIGS. 7B, 7F, and 7G, and 7C. In addition, some of the teeth 218 can contain an additional protrusion 284 on a square side edge 282 to assure that the capture gel holder 220 is placed into the electrophoresis cassette 190 in the proper orientation. FIGS. 6A, 7A, and 7F show such a protrusion. As indicated above, the teeth 238 are tapered, therefore the rounded and square edges 280 and 282 could possibly incorrectly fit through the slots 238 of the evaporative cover 194 a certain distance. The protrusions 284 limit such a possibility.

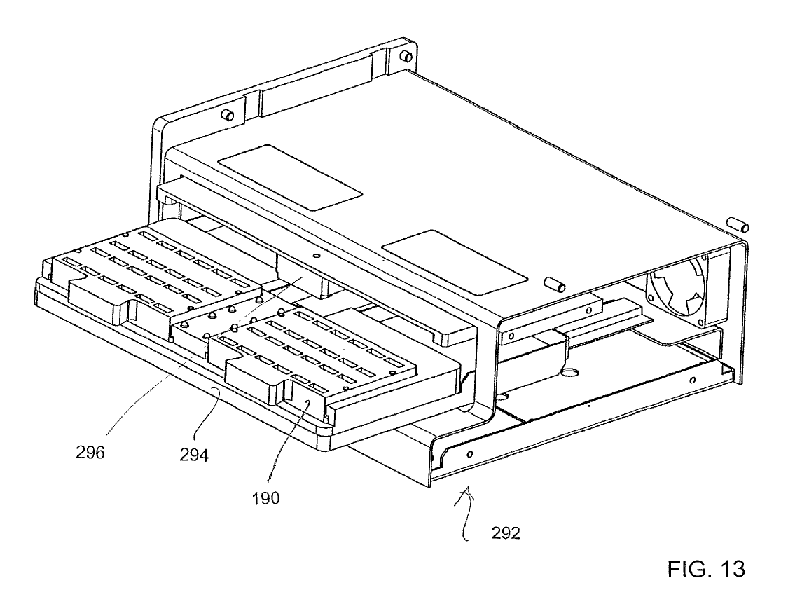

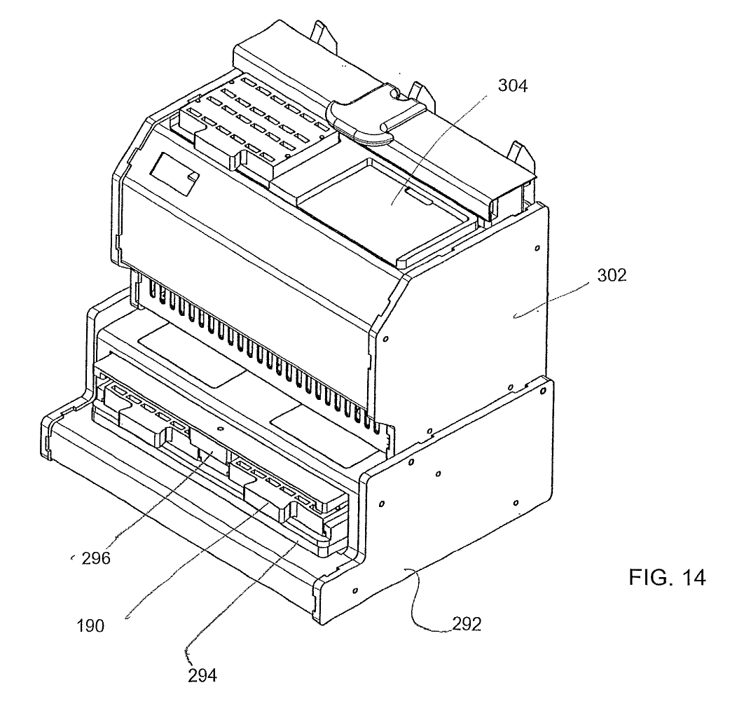

Referring back to FIGS. 5 and 6B, the capture gel holder 220 has a flat optical detector surface 288 on both sides of the part. These optical detector surfaces 288 are used in conjunction with an electrophoresis machine 292 described in conjunction to FIG. 13. While the capture gel holder 220 can only be placed in the cassette 190 in one position, optical detector surfaces 288 are located on both edges of the capture gel holder because of the two locations in the electrophoresis machine 292 that receives electrophoresis cassette 190.

In addition to the gel capture holder 220, a sample well forming comb 380 as shown in FIG. 8A and a molding comb 386 as shown in FIG. 8B are used with the electrophoresis cassette 190. The sample well forming comb 380 as shown in FIG. 8A has a plurality of teeth 382 which pass through the square openings 236 in the evaporative cover 194 to create sample wells in agarose or electrophoretic matrix 120 as described below. The molding comb 386 as shown in FIG. 8B is received in the enlarged slots 98 during the pouring of the agarose or electrophoretic matrix 120 as described below.

As indicated above, the method and apparatus allows rapid detection of target molecules in a biological sample. With the electrophoresis cassette 190 described and prior to a more detailed description of the capture gel 40 and of the electrophoresis cassette 190, a methods of detection using these items will be described.

Method

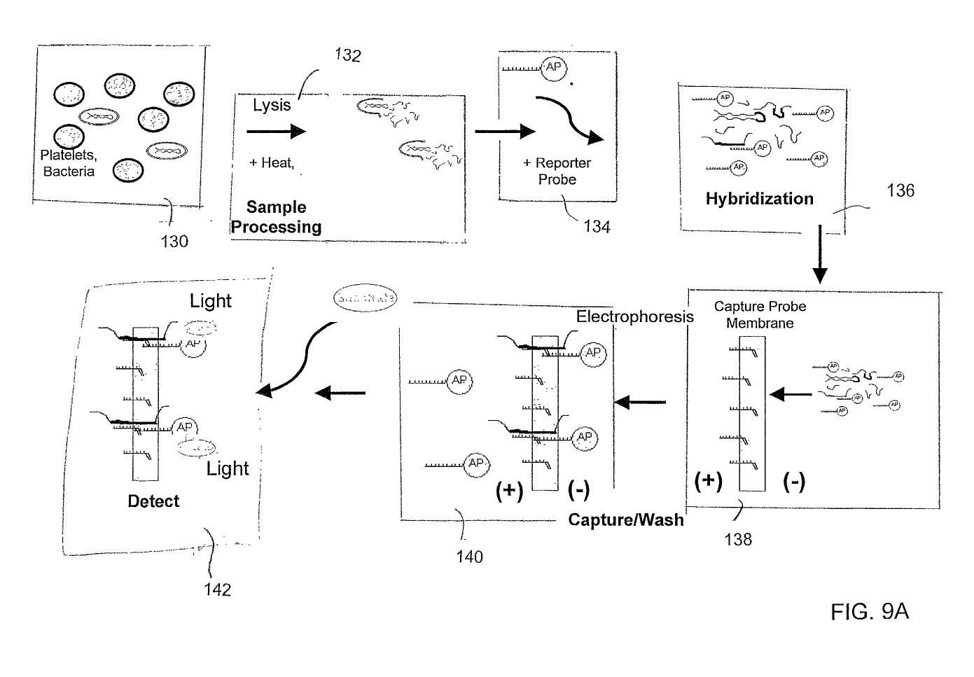

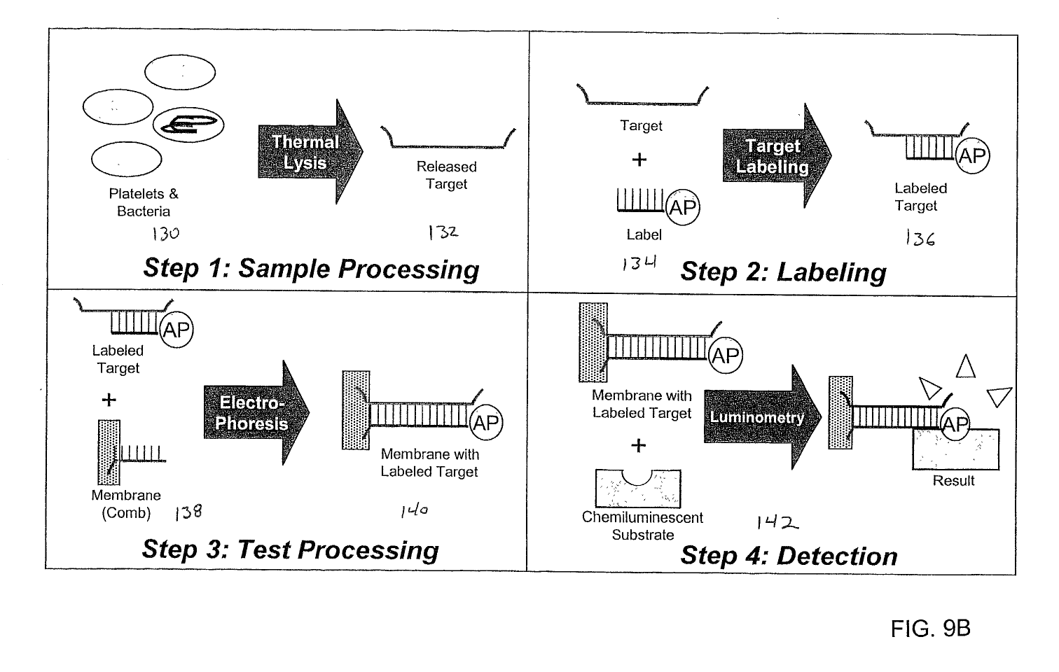

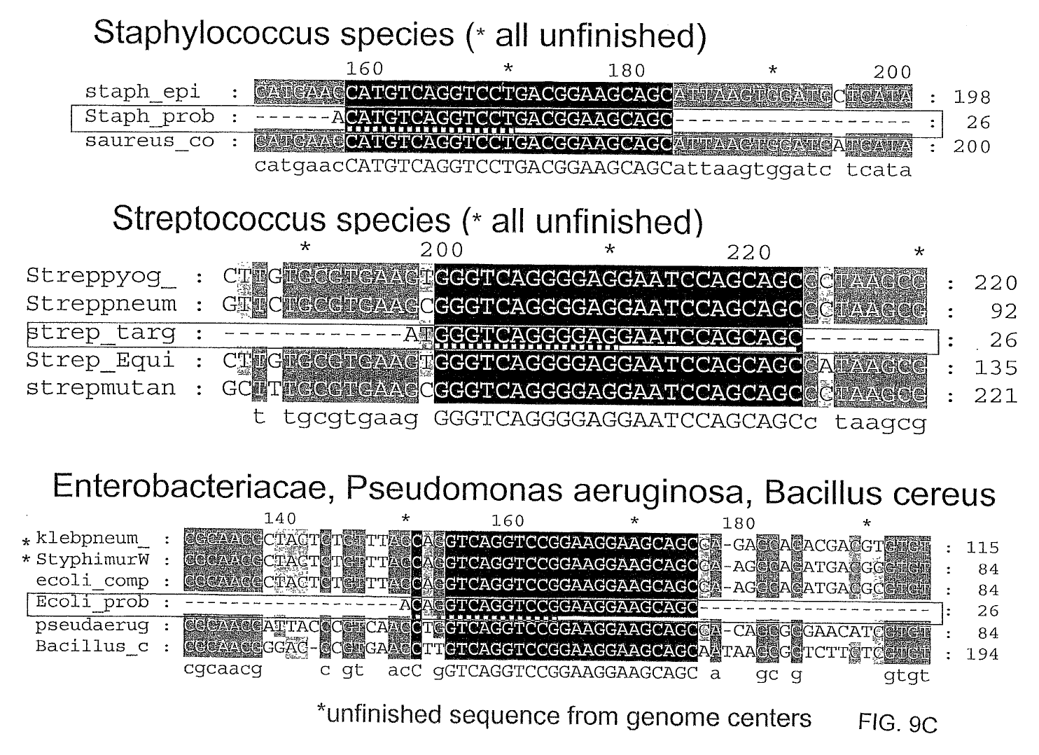

As described herein and illustrated by FIG. 9B, a biological sample believed to contain a target nucleic acid (either DNA or RNA) is obtained and processed in a manner (e.g., cells are lysed) to release the nucleic acids and render them suitable for detection. In one embodiment of the methods of the present invention, the released nucleic acids are contacted with a reporter probe, under conditions suitable for the reporter probe to hybridize to the target nucleic acids in the sample resulting in labeled (or tagged, e.g., detectably-labeled) sample nucleic acids. Optionally, an adapter probe can also be contacted with the sample nucleic acids under suitable conditions wherein the adapter probe also hybridizes to the sample nucleic acids. Thus the sample now comprises a mixture of labeled nucleic acids, unlabeled nucleic acids, and other components.

This sample mixture is then subjected to electrophoresis in the electrophoresis cassette described herein, where the mixture is brought into contact with the capture probe. The capture probe is specific for the target molecule contained in the sample (e.g., the target bacterial nucleic acid). If the sample contains the target molecule, e.g., the nucleic acid has a sequence that is complementary to that of the covalently bound capture probe sequence, the target molecule is immobilized to the capture probe in the thin gel of the gel holder. Due to the continued application of electrophoretic current, the unbound components of the sample continue to move through the thin gel and are separated from the bound components. In another embodiment of the claimed methods, the gel holder which contains the immobilized target/capture probe complex, can be transferred to a different slot in the electrophoresis cassette and the gel can be washed with buffer by briefly applying voltage which results in the unbound (e.g., unhybridized) components being removed from the gel. After separation, the bound components can be detected by a number of known methods. In one embodiment, detection of the bound target molecule is facilitated by moving the thin gel from the electrophoretic device to a detection device, such as a fluorescence or luminescence reader.

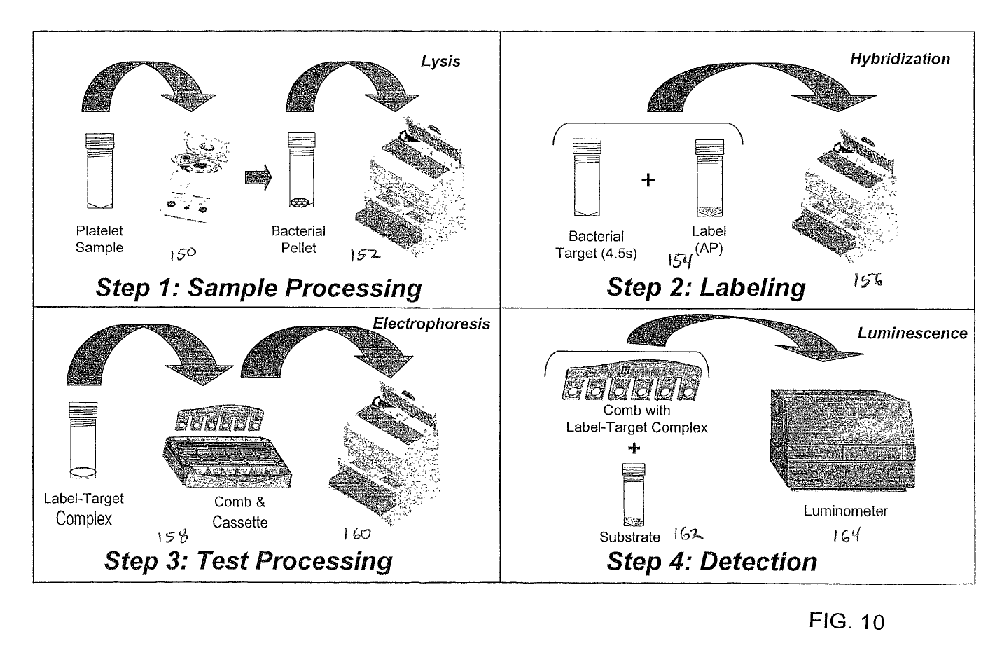

FIG. 9A shows the flow of molecules in the assay. FIG. 10 outlines schematically the steps for manipulating the sample and the reagent. Referring to text block 130 in FIGS. 9A and 9B and block 150 in FIG. 10, the sample which is suspected of containing the target analyte is prepared. Typically, the molecule to be detected is a target nucleic acid which is contained in a cell, bacterium, or virus. For example, as shown in FIG. 9C, conserved bacterial nucleic acid sequences can be used to detect a variety of bacteria present in a biological sample. As described herein, two probes can be used for each target RNA. One is the reporter probe that is used to label the target RNA. The other is the capture probe which is linked to the thin gel membrane. FIG. 9C shows the SRP RNA sequences (SEQ ID Nos. 1-17) from a panel of target organisms along with the complements of the probe sequences. The lines enclosed by the thin boxes indicates the DNA sequence that is complementary to the probes. (Sequence complements are shown so that the mispaired regions can be more easily identified.) The white dotted lines underline the sequences corresponding to the capture probes that are linked to the thin gel membrane. The solid white lines underline the sequences corresponding to the reporter probes. The capture probes are synthesized as 2′-O-methyl RNA oligonucleotides with a 5′-Acrydite (tm) modification (Acrydite (tm) is available commercially from Mosaic Technologies, Waltham, Mass.). The reporter probes are synthesized as 2′-O-methyl RNA oligonucleotides with a 5′-amino modification and are conjugated to an alkaline phosphatase enzyme label.

One of the first steps of sample processing is to lyse the cells to render the nucleic acids available for hybridization to a capture probe. For example, the sample is placed into a tube with a buffer or diluent and spun in a centrifuge at a speed and for a time sufficient for the cellular matter (e.g., platelets, cells, bacteria, viruses) to pellet in the tube. Such techniques are well-known to those of skill in the art. The supernatant is poured off the sample in the tube and the sample is resuspended in the rinse buffer and then spun down again as before. After spinning, the supernatant is poured off. A lysis buffer is then pipetted into the tube with the sample as represented by block 132 in FIG. 9A. Lysis buffer comprises a buffered solution comprising components sufficient for lysing the cellular matter and preserving the target nucleic acids in an undegraded state. For example, the lysis buffer can optionally comprise detergent or chaotrophic agents to facilitate lysis. Such buffers and components are well-known to those skilled in the art.

Referring to block 152 in FIG. 10, the sample is heated to about, for example, greater than about 100° C., more specifically, approximately 124° C.±4° C., for a time sufficient for the cells to lyse, e.g., for approximately five minutes, and then cooled to a specific temperature of less than 50° C., such as about 45° C. for about 2 to 6 minutes. In one embodiment, the sample is heated in the incubator unit 302 in FIG. 14. A reporter probe mixture is then introduced into the lysed sample. In one embodiment, the reporter probes comprise oligonucleotides which are conjugated to flourescent, phosphorescent chemiluminescent or enzymatic labels. Some examples of enzymatic labels include horseradish peroxidase or alkaline phosphatase. In general, enzymatic labels producing flourescent or chemiluminescent signals are used. The reporter probe is contacted with the lysed sample, as illustrated by block 134 in FIGS. 9A and 9B and block 154 in FIG. 10, under conditions sufficient for the reporter probe to hybridize to the nucleic acids in the sample, e.g., about 45° C.±2° C. for approximately 10 minutes, e.g., 9-11 minutes. For example, one reporter probe well-known to those of skill in the art is an alkaline phosphatase reporter probe. Thus, the sample hybridizes to the reporter probe as represented by block 136 in FIGS. 9A and 9B and 156 in FIG. 10, resulting in a labeled sample.

Referring to block 158 in FIG. 10 and block 138 in FIGS. 9A and 9B, the labeled sample is transferred into the sample wells of the electrophoresis cassette 190 shown in FIGS. 1-3. With the capture gel holder 220 located in the set of enlarged slots 98 which are closest to the positive (+) electrode of the migration channel 96, the voltage is applied to the electrophoresis cassette 190 cause the labeled sample to migrate through the electrophoresis matrix 120 from the location where it was introduced in the migration channel 96 away from one electrode 100 and toward the other electrode 100, as illustrated by block 140 in FIG. 9A and block 160 in FIG. 10. In some embodiments, the cassette is pre-warmed to a temperature of approximately 27-35° C. prior to electrophoresis. In one preferred embodiment, the electrophoresis temperature is approximately 31-35° C. In a preferred embodiment, voltage is adjusted from about 24-about 40 volts.

The capture probes/ligands may all have the same sequence or may have different sequences. It is recognized that each sequence in a thin gel can be localized in a specific area, for example of a specific tooth, or a spatially defined area within a single tooth. Therefore, each tooth of the capture gel holder can have a different capture probe/ligand for a different target molecule, or each tooth can contain multiple species of capture probes. In the case where multiple probes are present, each probe can be present in a unique spatially delineated area of the gel. Alternatively, the capture probes can be mixed together and the mixture dispersed throughout the capture gel.

After a set period of time, the voltage is turned off to the electrophoresis cassette 190 and the capture gel holder 220 is moved to the other enlarged segment 98, sometimes referred to as a wash slot. The voltage is turned on again and electrophoresis is continued for an additional time period, such as five minutes. The current is in the same direction during the second or wash period. By moving the capture gel holder 220 to the second enlarged segment 98 the migration of the sample is away from the capture gel 40 and thus any unbound sample on the capture gel holder 220 will pass through and no new sample will come in contact with the capture gel 40. Only the specifically bound target remains on the comb.

Referring to block 142 in FIGS. 9A and 9B and block 162 in FIG. 10, the capture gel holder 220, such as the comb shown in FIG. 5, is removed from the electrophoresis cassette 190 with the capture gel 40 with the labeled target nucleic acid hybridized to the capture probes of the capture gel matrix. The capture gel holder 220 is then placed in a buffer solution and the substrate specific for the reporter probe of the labeled sample is contacted with the sample for a time and at a temperature sufficient for the reporter probe to interact with the substrate to generate a detectable signal. Then the capture gel holder 220 is placed in a reader as represented by block 164 in FIG. 10 so that the signal, such as chemiluminescence, is detected. Presence of a detectable signal is indicative of the presence of the target molecule, e.g. nucleic acid, in the sample. The detectable signal is produced by the label of the reporter probe, which is the probe shown in block 134 of FIG. 9A or block 154 of FIG. 10.

Samples

Any biological sample can be analyzed using the methods and devices described herein. The methods of the present invention are applicable to analysis of any chemical entity that can be electrophoresed (e.g., a charged molecule that has detectable mobility when placed in an electrophoretic field) and that binds to, or is bound by, nucleic acids. Such entities include, for example, DNA or RNA samples, nucleic acid binding proteins, and aptamer binding partners (aptamers are nucleic acids that are selected to bind to specific binding partners such as peptides, proteins, drugs, polysaccharides and small organic molecules, e.g., theophylline and caffeine (Jenison, et al., Science, 263:1425-1429 (1994)). For example, methods described herein can be used for analysis and purification of target nucleic acids using immobilized capture probes, where specific binding involves base pairing interactions between sample nucleic acids and the capture probe, as in nucleic acid hybridization. The methods described herein are also useful for purification of sequence-specific nucleic acid binding proteins, since synthetic nucleic acids of defined sequence can be immobilized in matrices commonly used for protein electrophoresis.

The test sample can be from any source and can contain any molecule that can form a binding complex with a capture probe. Specifically encompassed by the present invention are samples from biological sources containing cells, or platelets, obtained using known techniques, from body tissue (e.g., skin, hair, internal organs), or body fluids (e.g., blood, plasma, urine, semen, sweat). Other sources of samples suitable for analysis by the methods of the present invention are microbiological samples, such as viruses, yeasts and bacteria; plasmids, isolated nucleic acids and agricultural sources, such as recombinant plants.

The test sample is treated in such a manner, known to those of skill in the art, so as to render the target molecules contained in the test sample available for binding or hybridizing. For example, if the target molecule is a nucleic acid present in a cell, a cell lysate is prepared, and a crude cell lysate (e.g., containing the target nucleic acid as well as other cellular components such as proteins and lipids) can be analyzed.

Alternatively, the target nucleic acids can be isolated (rendering the target nucleic acids substantially free from other cellular components) prior to analysis. Isolation can be accomplished using known laboratory techniques. The target nucleic acid can also be amplified (e.g., by polymerase chain reaction or ligase chain reaction techniques) prior to analysis.

Probes

Capture probes are ligands, or molecular sequences complimentary to a predicted analyte immobilized in an electrophoretic gel matrix by covalent attachment to a polymer suitable for use as an electrophoretic medium. A variety of capture probes can be used in the methods of the present invention. Typically, the capture probes of the present invention comprise a nucleic acid with a nucleotide sequence substantially complementary to the target molecule wherein the target molecule hybridizes to the capture probe. The complementarity of nucleic acid capture probes need only be sufficient to specifically bind the target molecule and demonstrate the presence or absence of the target molecule. Probes suitable for use in the present invention comprise RNA, DNA, nucleic acid analogues, and chimeric probes of mixed class comprising a nucleic acid with another organic component, e.g., peptide nucleic acids. Capture probes can be single-stranded or double-stranded nucleic acids.

As defined herein, the term “nucleic acid” includes DNA or RNA. Nucleic acids referred to herein as “isolated” are nucleic acids separated away from the components of their source of origin (e.g., as it exists in cells, or a mixture of nucleic acids such as a library) and may have undergone further processing. Isolated nucleic acids include nucleic acids obtained by methods known to those skilled in the art. These isolated nucleic acids include substantially pure nucleic acids, nucleic acids produced by chemical synthesis, by combinations of biological and chemical methods, and recombinant nucleic acids which are isolated.

As defined herein, “substantially complementary” means that the nucleotide sequence of the capture probe need not reflect the exact nucleotide sequence of the target molecule, but must be sufficiently similar in identity of sequence to hybridize with the target molecule under specified conditions. For example, non-complementary bases, or additional nucleotides can be interspersed in sequences provided that the sequences have sufficient complementary bases to hybridize therewith.

Specified conditions of hybridization can be determined empirically by those of skill in the art. For example, conditions of stringency should be chosen that significantly decrease non-specific hybridization reactions. Stringency conditions for nucleic acid hybridizations are explained in Current Protocols in Molecular Biology, Ausubel, F. M., et al., eds., Vol. 1, Suppl, 26, 1991, the teachings of which are herein incorporated by reference, in their entirety. Factors such as probe length, base composition, percent mismatch between the hybridizing sequences, temperature and ionic strength influence the stability of nucleic acid hybrids. Stringent conditions, e.g., moderate, or high stringency, can be determined empirically, depending in part on the characteristics of the probe and target molecule.

Typically, the length of a capture probe will be at least 5 nucleotides in length, more typically between 5 and 50 nucleotides, and can be as long as several thousand bases in length.

Probes comprising modified nucleotides may also be useful. For instance, nucleotides containing deazaguanine and uracil bases may be used in place of guanine and thymine-containing nucleotides to decrease the thermal stability of hybridized probes (Wetmur, Critical Reviews in Biochemistry and Molecular Biology, vol. 26, pp. 227-259, 1991). Similarly, 5-methylcytosine can be substituted for cytosine if hybrids of increased thermal stability are desired (Wetmur, Critical Reviews in Biochemistry and Molecular Biology, vol. 26, pp. 227-259, 1991). Modifications to the ribose sugar group, such as the addition of 2′-O-methyl groups can reduce the nuclease susceptibility of immobilized RNA probes (Wagner, et. al. Nature, 372: 333-335, (1994)). Modifications that remove negative charge from the phosphodiester backbone can increase the thermal stability of hybrids (Moody, et. al., Nucleic Acids Res., 17: 4769-4782 (1989); Iyer et. al., J. Biol. Chem., 270: 14712-14717 (1995)).

Nucleic acid analogues can also be useful as immobilized probes. One example of a useful nucleic acid analogue is peptide nucleic acid (PNA), in which standard DNA bases are attached to a modified peptide backbone comprised of repeating N-(2-aminoethyl)glycine units (Nielsen, et. al., Science, 254: 1497-1500, (1991)). The peptide backbone is capable of holding the bases at the proper distance to base pair with standard DNA and RNA single strands. PNA-DNA hybrid duplexes are much stronger than equivalent DNA-DNA duplexes, probably due to the fact that there are no negatively charged phosphodiester linkages in the PNA strand. In addition, because of their unusual structure, PNAs are very resistant to nuclease degradation. For these reasons, PNA nucleic acid analogues are useful for immobilized probe assays. It will be apparent to those skilled in the art that similar design strategies can be used to construct other nucleic acid analogues that will have useful properties for immobilized probe assays.

As used herein a “label” is any means for distinguishing a capture probe having an analyte bound thereto. For example, the label may be a molecule complementary to the capture probe—an analyte complex where the label has been provided with a radio-isotope, a fluorescent, a chemiluminescent, or similar chemical tag such as used in a sandwich hybridization assay. Alternatively, the label may be an enzyme capable of interacting with a substrate to produce a detectable signal. A signal probe can be used to provide a label on the capture probe-analyte complex.

Optionally, each capture probe in a thin gel or polymeric gel is localized in a specific area. A plurality of thin gels having differing capture probes/ligands can be arranged to form arrays so all the probes in a particular area have a known sequence. The thin gel or polymeric gel is further described below.

Methods for covalently attaching capture polymers to polymerizable chemical groups have been developed. For example, methods for covalently attaching nucleic acids to polymerizable chemical groups are found in U.S. Pat. No. 5,932,711 (Boles); Rehman, et.al., Nucleic Acid Res., 27:649-655 (1999); U.S. patent application Ser. No. 08/971,845 filed Aug. 8, 1997; U.S. patent application Ser. No. 09/285,380 filed Apr. 2, 1999; U.S. patent application Ser. No. 09/286,091 filed Apr. 2, 1999; U.S. Provisional Patent Application No. 60/151,267 filed Aug. 27, 1999; and U.S. Provisional Patent Application No. 60/177,844 filed Jan. 25, 2000, the disclosures of which are incorporated herein in their entirety by reference (See also, Kenney, Ray, and Boles, Bio Techniques 25:516-521 (1998), the disclosure and teachings of which are incorporated herein in their entirety.) Capture probes can also be covalently attached to a matrix using a thiol attachment chemistry as described in U.S. Provisional Patent Application No. 60/151,267 filed Aug. 27, 1999, U.S. Provisional Patent Application No. 60/177,844 filed Jan. 25, 2000, and U.S. patent application Ser. No. 09/649,637 titled “Methods of Immobilizing Ligands on Solid Supports and Apparatus and Methods of Use Therefor” by Abrams, et al., filed Aug. 28, 2000, the teachings of which are incorporated herein in their entirety. Capture probes are attached to a polymeric material that provides a sieving function in order to increase contact with the capture probe. Thin gel matrices may also include spacer molecules and matrix strengthening molecules.

Nucleic acids, modified nucleic acids and nucleic acid analogs can also be coupled to agarose, dextrans, cellulose and starch polymers using cyanogen bromide or cyanuaric chloride activation. Polymers containing carboxyl groups can be coupled to capture probes provided with a primary amine group using carboiimide coupling. Polymers having primary amine groups incorporated therein can be coupled to amine-containing capture probes with glutaraldehyde or cyanuric chloride. Many polymers can be modified with thiol-reactive groups which can be coupled to thiol-containing synthetic capture probes. Representative protocols and other examples can be found in Cass and Ligler (eds.), “Immobilized Biomolecules in Analysis: A Practical Approach,” 1998, Oxford University Press, Oxford, UK, and Hermanson, Mallia, and Smith, “Immobilized Affinity Ligand Techniques, 1992, Academic Press, San Diego, Calif., the disclosures of which are incorporated by reference.

Adaptor probes/molecules can also be used in the methods of the present invention. Such probes are described in U.S. patent application Ser. No. 09/285,380 and PCT/U.S.00/08529, the teachings of which are herein incorporated by reference.

Gel Matrix

One of the elements of the invention is the capture gel 40, such as a polymeric gel or a thin gel. This capture gel matrix is used in conjunction with the probe or capture probe, to capture the target molecule (i.e., desired component).

The capture gel refers to the gel framework to which complementary sequences are attached. Examples of materials suitable for use in forming the gel matrix include both gel-forming and non-gel forming polymers. Any gel matrix suitable for electrophoresis can be used for preparation of the gels of the present invention. Suitable matrices include acrylamide and agarose. It is recognized that other materials can be used as well for the capture gel. Examples include chemically modified acrylamides, starch, dextrans and cellulose-based polymers. Additional examples include modified acrylamides and acrylate esters (for example see Polysciences, Inc. Polymer & Monomer catalog, 1996-1997, Warrington, Pa.), starch (Smithies, Biochem. J., 71:585 (1959); product number S5651, Sigma Chemical Co., St. Louis, Mo.), dextrans (for examples see Polysciences, Inc. Polymer & Monomer catalog, 1996-1997; Warrington, Pa.), and cellulose-based polymers (for example see Quesada, Current Opinion in Biotechnology,8:82-93 (1997)). Any of these polymers listed above can be chemically modified to allow specific attachment of capture probes for use in the present invention. Other suitable methods can be found in the literature (For review, see Wong, “Chemistry of Protein Conjugation and Cross-linking”, CRC Press, Boca Raton, Fla., 1993). Incorporation of a reinforcement into the thin gel matrix facilitates the handling of the capture gel.

Composite matrices of polymers are also useful for forming the thin gels of the present invention. A composite matrix contains a mixture of two or more matrix forming materials, such as for example acrylamide-agarose composite gels. These gels typically contain 2-5% acrylamide and 0.5%-1% agarose. In these gels, the acrylamide concentration determines the functional pore size. Agarose provides mechanical strength without significantly altering the gel pore size of the acrylamide.

Referring to FIG. 11A, the capture gel 40 has non-conductive mesh 42 encapsulated in a polymer gel 44. A plurality of capture probe molecules are covalently bound to the polymer gel 44 throughout the three dimensional capture gel, which fills the spaces 48 of the fibers 50 of the mesh 42, as best seen in FIG. 11B. The non-conductive mesh 42 is seen to be substantially entrapped in bubble-free capture (thin) gel 40. In the cross-sectional view of FIG. 11B, a top surface 52 and a bottom surface 54 of the capture gel (reinforced thin gel) 40 are shown substantially flat. While the top surface 52 and the bottom surface 54 are illustrated as substantially flush with the top surface and the bottom surface of the non-conductive mesh 42, respectively, either or both of the top surface 52 and the bottom surface 54 of the capture gel 40 can be distanced from their respective surfaces of the mesh 42.

In one embodiment, the polymer gel 44 is an acrylamide gel. The type of capture probe molecules are dependent on what is desired to be capture. For example, the capture probe molecules can be an oligonucleotide carrying reactive ethylene groups.

The capture gel 40 with the non-conductive mesh 42 can be formed by various methods. One method of forming the capture gel 40 is by capillary action. Porous material, a non-conductive mesh, is placed between two glass plates so that the material extends from between the two plates while being supported by one of the plates. Gel-forming solution is applied to an edge of the material that extends from under the top glass plate. The gel-forming polymer solution is drawn into the material by capillary action and between the two glass plates. Plate spacers may be used between the glass plates to allow an increase in the thickness of the layer when gelled. Preferably, a releasing agent is applied to the mold, be it glass or some other material, in order to facilitate release of the molded and reinforced thin gel.

An alternative method is for porous material to be placed in a mold and a gel-forming polymer solution, having ligand covalently bound to the polymer providing sieving properties to the gel, can be added to the mold and allowed to solidify. Air bubbles within the gel matrix should be avoided, for example by forming the thin gel under pressure or by vibrating the mold.

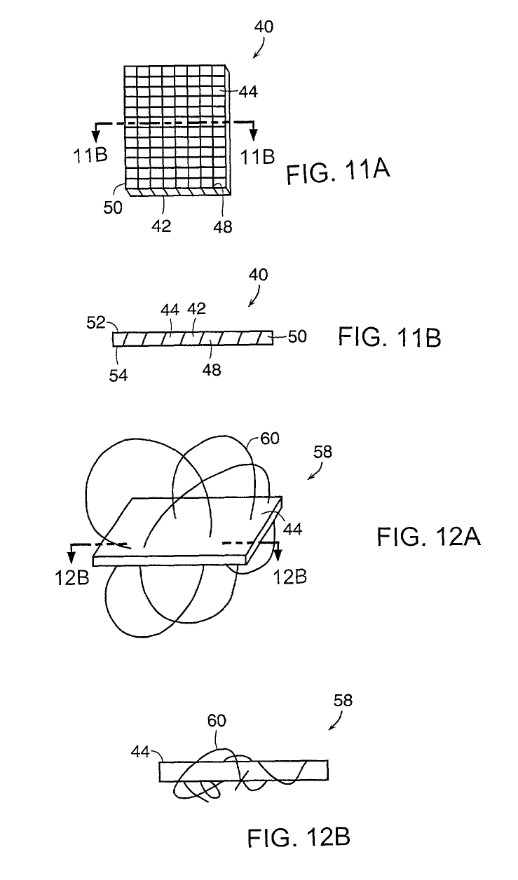

Referring now to FIG. 12A and FIG. 12B, an alternative embodiment of a reinforced capture (thin) gel 58 having a polymer gel 44 encased within a fibrous glass fiber infra-structure 60 is illustrated. The glass fiber reinforcement 60 is seen to extend above and below the surfaces of the polymer gel 44. Capture probe molecules are covalently bound to polymers 44 throughout the three dimensional capture gel 58.

Preferably, the capture gel 40 of the present invention has sufficient mechanical strength to allow its removal from the electrophoresis device for washing and detection procedures, such as described below. However, the capture gel can be reinforced either by mechanical or molecular means. Examples of an internal structural reinforcement for such a thin gel matrix include a variety of non-conductive materials having pores. Such materials include for example mesh, screen or honeycomb material; non-woven fibrous material such as glass fiber and mat; and woven or knitted material such as fabric. Examples of materials having pores comprise mesh, woven, and non-woven materials. Mesh materials include polyester silk screening material such as is available from Sefar America, Inc., Briancliff Manor, NJ. Corp., polyamide resin mesh, nylon mesh, and polyethylene or polypropylene honeycomb material. Woven materials include loosely woven cloth such as for example J-Cloth™ available from Johnson & Johnson Ltd. Non-woven materials include membranous materials comprising cellulose esters such as cellulose acetate butyrate, nitrocellulose, cellulose propionate such as described in U.S. Pat. No. 4,006,069 (Hiratsuka, et.al.), the disclosure of which is incorporated herein by reference. Where the reinforcement is to be internal to the thin gel matrix and there is to be a layer of gel on both sides of the reinforcing mesh or cloth so that the reinforcement material is encased by, and enmeshed with, the gel, the thickness of the gel is preferably not greater than that which can be supported by the mesh or cloth. For embodiments where the gel is to be retained predominantly on the surface of the reinforcement material, it is preferred that the surface of the reinforcement material is either rough, irregular, porous or otherwise capable of allowing the gel to interpenetrate the material. Some examples of three-dimensionally stable reinforcement materials are porous materials such as porous particles made by a method as disclosed in British Patent No. 1421 531 (UKAEA), the disclosure of which is incorporated herein by reference in its entirety. Other examples of reinforcement materials include thinly sectioned sponges. Also other contemplated reinforcements include porous materials that are coated to enhance the adhesion of the gel matrix to the porous material such as coated polyester, as described in U.S. Pat. No. 5,672,416 (Radola), the disclosure of which is incorporated herein by reference in its entirety.

The capture gel 40 can also include spacer molecules and matrix strengthening molecules. As used herein a “spacer molecule” is a structure capable of polymerizing with or binding to at least one of the gel matrix forming polymer molecules and functions to distance a moiety bound to the spacer from the gel matrix forming polymer backbone. Optionally, the spacer molecule is capable of binding with any two of the gel matrix forming polymeric components and can thereby affect the density and/or spatial orientation of the capture probes bound to a gel matrix forming polymeric component in any particular region of the thin gel matrix by distancing one polymer from another polymer in the matrix. Additionally, spacer molecules may provide reactive sites for binding to a moiety or molecule other than the matrix forming polymers. For example, the spacer molecule may provide binding sites for polyethylene glycol or a detergent.

As used herein “matrix strengthening polymers” are polymers useful for cross-linking, stiffening, or otherwise modifying the gel matrix to make it more durable during handling, without adversely affecting the desired sieving and/or analyte binding properties of the resulting thin gel matrix. Matrix strengthening polymers toughen the thin gel matrix increasing its mechanical strength thus allowing it to be handled without additional external reinforcement. Exemplary polymers include agarose. In addition to strengthening the thin gel matrix, this polymer component may also provide additional sites for coupling gel modifying constituents. For example, chemical groups such as methyl or hydroxyl groups may be added to modify the hydrophobic/hydrophilic nature of the gel. Sulfate or quaternary amine groups may be added to introduce ionic groups into the thin gel.

Method of Preparing Cassette With the electrophoresis cassette 190 described briefly, the process of assembly of the electrophoresis cassette 190 is described in greater detail. The stainless steel or platinum strips 206 are placed the electrode channels 92 and 94 seen in FIG. 1 of the base 192. The strips 206 have curved ends to receive the posts 204 as seen in FIG. 3 as explained below and the slots 200 in the ribs 198 ensure the strips are installed properly.

With the electrode strips 206 in position, the evaporative cover 194 as seen in FIGS. 4A and 4B is positioned on the base 192. As indicated above, the cover 194 and the base 192 have tabs 248 and notches 250 to ensure proper installation. In one embodiment, the evaporative cover 194 and the base 192 are welded together by an ultrasonic process.

The four posts 204 associated with the two electrodes 202 are each inserted into one of the holes 246 in the evaporative cover 194. The bore 210 in the base 192, as seen in FIG. 1 receives a portion of the cylindrical rod portion of the post 204 with the rod portion passing through the curved end of the strips 206. The circular disk portion 212 of the post 204 overlies the top of the evaporative cover 194.

With the evaporative cover 194 and the base 192 of the electrophoresis cassette 190 assembly, the teeth of a pair of molding combs are inserted through the slots 238 in the evaporative cover 194 and into the enlarged slots 98, as seen in FIG. 1. One of the identical molding combs 386 is seen in FIG. 8B. Both sets of enlarged slots 98 receive teeth of the molding combs. Alternative to using a pair of molding combs 386 of FIG. 8B, a single molding comb 390 having two parallel sets of teeth such as seen in FIG. 8C, can be used.

With the teeth of molding comb(s) 386 or 390 in position in the enlarged slots 98 of the migration channels 96, an electrophoretic matrix 120 is dispensed in the base 192 through the sample openings and fills a portion of the migration channel 96 to a predetermined height. In one embodiment, the electrophoretic matrix 120 can be formed from gel forming polymers such as agarose or starch. In one embodiment, the agarose is formed by using a molten solution of 1% agarose in 0.1×TrisBP. The following is mixed 100 ml 0.1×TrisBP buffer with 1 g agarose powder (Sigma A9539 General Use Molecular Biology Grade). The procedure is repeated for all the migration channels 96, and a sample well comb 380 is inserted into the cassette. The sample well comb is shown in FIG. 8A.

Molten agarose solution is dispensed into channels 96 through sample opening 236 in evaporative cover, taking care to reserve enough empty space to allow insertion of sample well comb.

For illustrative purposes, the agarose or electrophoretic matrix 120 is shown in FIG. 1 filling one migration channel 96. A sample well 174 is shown in the agarose 120. As indicated above, the evaporative cover 194 is welded to the bore 192 prior to the dispensing of the agarose 120 into the channels 96.

For the embodiment shown in FIGS. 1-3, approximately 1.4 m. agarose solutions are used per channel. After fitting all 6 channels 96, the sample well comb shown in FIG. 8A is inserted into the sample openings 236, and the agarose is allowed to cool. After cooling, the molding and sample-well combs are removed, and the electrode channels 92 and 94 and the sample wells 174 within the channels 96 are filled with electrophoresis running buffer through cover openings 238 and 236, respectively. In addition, wash buffer is placed in wash wells 222 and conditioning buffer is placed in conditioning wells 224.

With the agarose in the electrophoresis cassette 190 with the sample wells formed and the proper buffers in the proper places, the electrophoresis cassette 190 can be foiled sealed for shipping in one embodiment. The capture gel holder 220 is sealed separately and contains the desired capture probes in one embodiment.

The cassettes of the present invention can be disposable (i.e., used once and discarded) or reused after a suitable cleaning process. For instance, in embodiment utilizing alkaline phosphate (AP) as a label, the following treatment can be used to remove trace amounts of AP. The parts of the electrophoresis cassette 190 are soaked in a solution of 1% hydrochloric acid and 1 millimolar (mM) EDTA (ethylenediaminetetraacetic acid) for at least 30 minutes to remove residual reagents. The parts or components are then washed thoroughly with water and air-dried on a clean surface.

When detection of a target molecule is desired, the electrophoresis cassette 190 and capture gel holder 220 are unsealed if sealing step had occurred. A sample is also prepared as described with reference FIGS. 9A, 9B and 10. A hybridization mixture is loaded into the sample wells 174 in the electrophoresis cassette 190 through the square openings 236 in the evaporative cover 194.

The capture gel holder 220 which contains the capture gel 40 with the desired capture probes is inserted in the electrophoresis cassette so that the teeth 218 enter the first set of enlarged slots 98 in the migration channel 90. The first set of slots 98 is that set closest to the (+) electrode.

The electrophoresis cassette 190 is placed in an electrophoresis machine 292. Referring to FIG. 13, the electrophoresis machine 292 for receiving the electrophoresis cassettes 190 is shown. In FIG. 13, for illustration purposes one of the sides of the electrophoresis machine 292 is removed. The electrophoresis machine 292 has a tray 294 which slides out of the machine and receives the electrophoresis cassettes 190. The machine 292 has electrical connections in the center of the electrophoresis machine 292 in a bar 296 such that when the tray 294 slid in to the machine 292, the bar 296 in the center engages the cylindrical disc 212 of the post 204 of the electrodes 202. Only one side of each of the electrodes 202 is contacted. The purpose for having a pair of posts 204 for each electrode 202 is such that the electrophoresis cassettes 190 may be placed in either spot on the tray 294 in the electrophoresis machine 292.

The electrophoresis machine 292 has timing features and temperature sensors to ensure that the electrophoresis cassette 190 does not exceed a certain temperature. In addition, with respect to the capture gel holder 220, the optical detector surface 288 is used in conjunction with the electrophoresis machine 292 such that machine 292 does not operate without a capture gel holder 220.

Referring to FIG. 14, the electrophoresis machine 292 is shown with an incubator unit 302 overlying it. The incubator unit 302 has a pair of warming blocks 304 for receiving the electrophoresis cassettes 190. The blocks 304 are used for prewarming cassettes 190 to proper running temperature prior to electrophoresis.

The electrodes 100 of the electrophoresis cassette 190 of FIG. 2 are connected to a DC power supply in the electrophoresis machine 292 and a constant voltage is applied for a set time period. Temperature will increase during the run, but should not be allowed to increase above a temperature which will impair hybridization of the probes with the target nucleic acid. In a preferred embodiment the cassette temperature is controlled within the electrophoresis machine 292.

Referring to block 158 of FIG. 10, after a set period of time of migration of the sample on the electrophoretic matrix 120 towards the capture gel 40 in the capture gel holder 66, the power is turned off. The capture gel holder 66 is moved from the enlarged segment 98 in the migration channel 96 to the other enlarged segment 98 in the same electrophoresis cassette 86 which is proximal to the (−) electrode. Prior to the movement to the other enlarged segments 98, the teeth of the capture gel holder 220 are placed into the set of wash wells 222 that contains the wash buffer. In one embodiment, the buffer is the wash buffer of example 1 and the capture gel 40 on the teeth 218 are in the buffer for 5 seconds.

The electrodes 100 are connected to a DC power supply and a constant voltage is applied for a set time period. Once again, care is taken not to allow the temperature of the cassette to exceed a temperature which will impair hybridization of the probes.

As indicated above, when discussing the method in relation to FIG. 10, the current is in the same direction during the second or wash period. By moving the capture gel holder 220 to the second enlarged segment 98, the propagation of the residual sample which may be trapped in the channel 96 is away from the capture gel 40 of the capture gel holder 220 and thus any sample on the capture gel holder 220 that will not be captured by the capture probe will pass through and no new sample will come in contact with the capture gel 40.

After the above steps, the detection of captured nucleic acid target-alkaline phosphatase conjugated probe complex on the thin gel membranes, the capture gel 40 of the capture gel holder 220, is accomplished. The capture gel holder 220 is removed from the wash or second set of slots 98 of the electrophoresis cassette. The teeth 218 with the capture gel 40 are placed in a conditioner buffer in the second set of wash wells 224. The capture gel 40 is soaked in the solution and incubated at room temperature for 5-10 minutes on the incubator unit of FIG. 14.

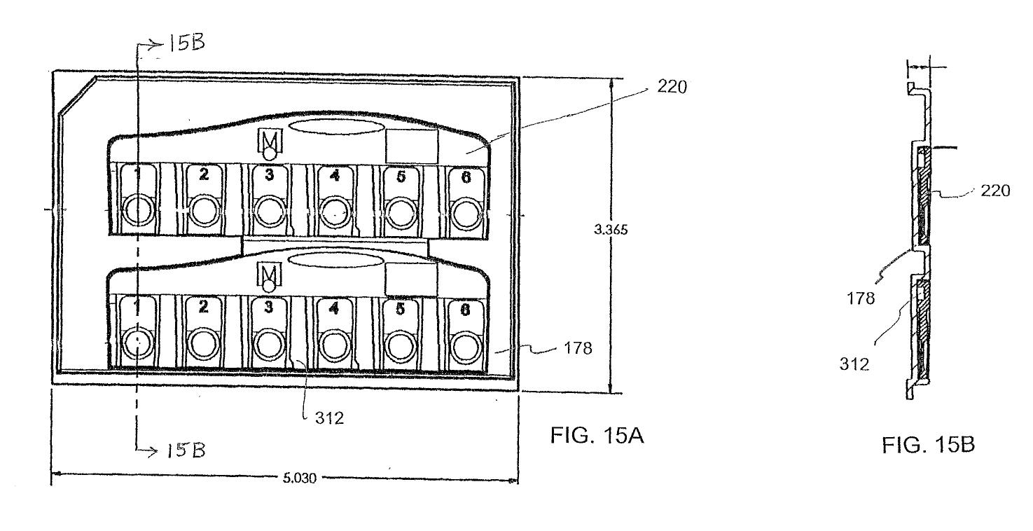

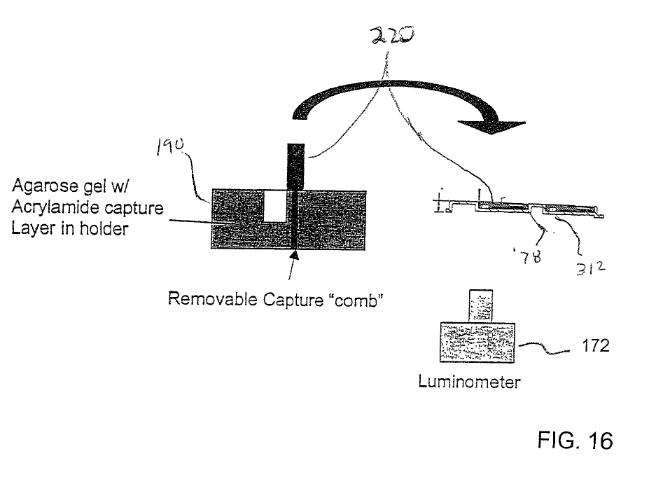

Referring to FIGS. 15A and 15B, the detection tray 178 for receiving the capture gel holders 220 is placed into a detector 172 such as a luminometer of FIG. 16. The detection tray 178 has a recess 312 for receiving a pair of capture gel holders 220. In one embodiment, the recess 312 is shaped to fit the capture gel holder 220 described above. The detection tray 178 is sized in one embodiment to be approximately the size of a 96-well type tray plate in length and width. In this embodiment, the detection tray 178 is not as tall as a conventional 96-well type tray plate. Therefore, it has a length of approximately 5.03 inches and a width of 3.365 inches.

The capture gel holder 220 is then placed in a detection tray 178, as shown in FIGS. 15A & 15B. The capture gel holder 66 and the detection tray 178 are placed in a microplate luminometer 172 such as a Walla Victor 2 sold by E.G.&G Walla of Turku, Finland, as represented in FIG. 16. Relative light units (RAU) are read and both the RAU and signal to noise ratio=(Test sample membrane minus empty well)/(Negative control sample membrane minus empty well) are reported.

Method of Purification

In another embodiment of the present invention, target nucleic acids are purified by contact with the capture probe covalently attached to a thin gel matrix. For example, after the target nucleic acid is bound to the capture probe, and separated away from the remaining components of the sample, the bound target nucleic acid can be released from the probe by changes in conditions (e.g., increased temperature) and the released target nucleic acids can be collected into a clean receptacle for further processing. Such purification can be performed using capture probes covalently bound to thin gel membranes where the gel membranes are contained in any suitable holder or support device, e.g. a comb such as described herein or micro liter plates, such as the 96-well micro liter well plates well-known to those of skill in the art.

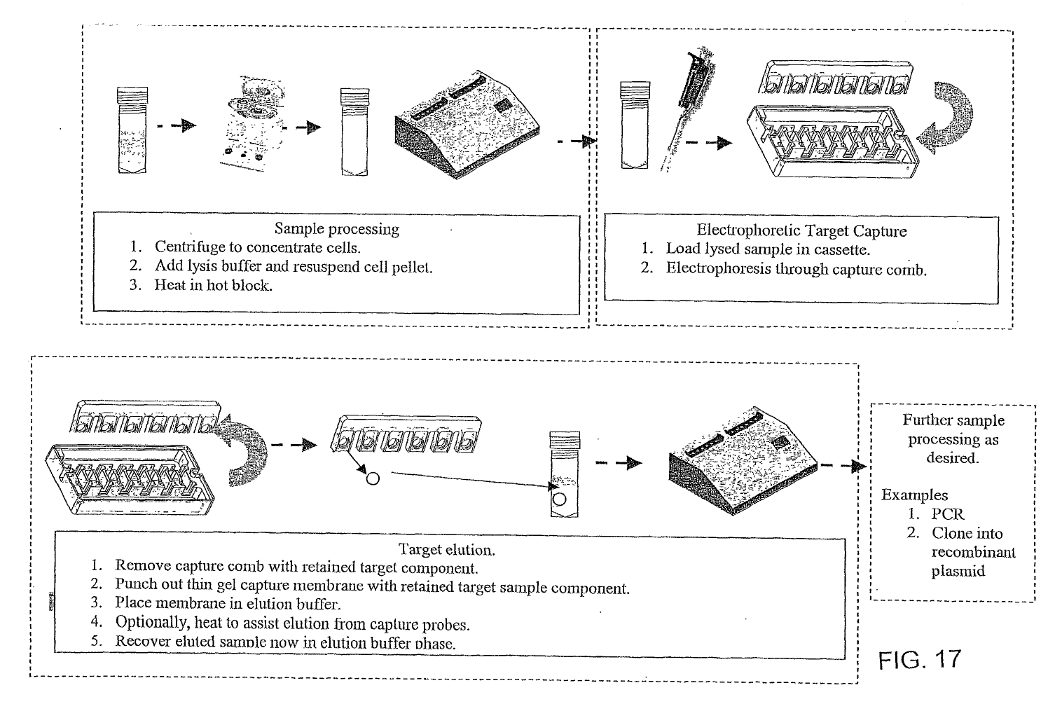

For example, referring to FIG. 17, the purification steps are similar to the method shown in FIG. 10 and discussed above until the detection step, block 162 and 164 in FIG. 10 After the desired target molecule is captured and immobilized to the capture probe in the gel, the capture gel holder 220 is removed from the electrophoresis cassette 190 The capture gel holder 220 is subjected to conditions sufficient to break the bonds between the capture probe and the target molecule, thereby releasing the target molecule from the probe. In the embodiment shown in FIG. 17, the thin gel membrane is removed from the holder and placed in a tube with elution buffer. The tube is heated to elute the hybridized target from the capture probes. The released target molecules can be recovered using techniques well-known to those skilled in the art and then used in further procedures, such as, for example, PCR amplification.

Alternative Cassette and Holder

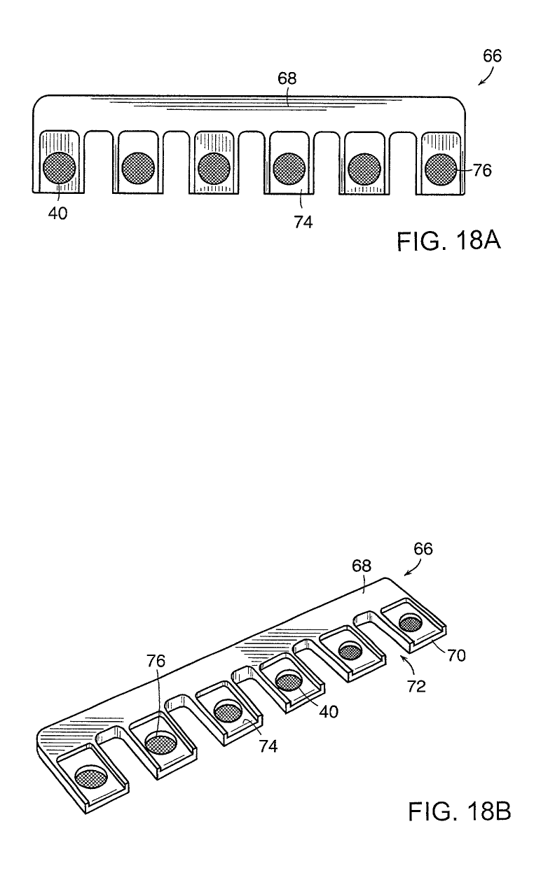

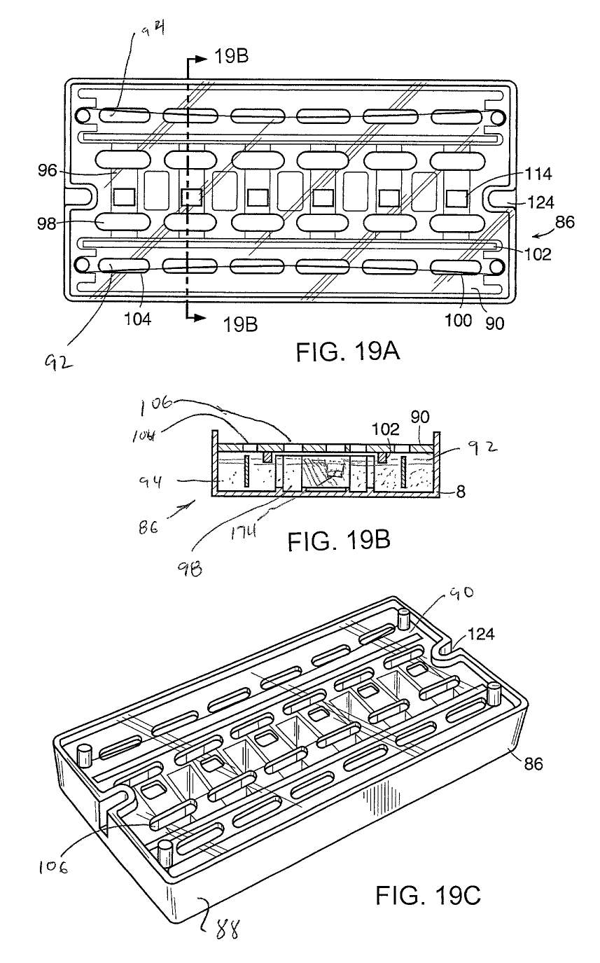

An alternative capture gel holder 66 is seen in FIGS. 18A and 18B. The capture gel holder, also sometimes referred to as a comb or sample comb, 66 has a handle 68 and a plurality of teeth 70.

Each of the teeth 70 has a thinned central region 74 in which is located a bore 76. The bore 76 extends through the tooth 70. The thinned central region 74 facilitates bubble removal as explained above with respect to the first embodiment. The non-conductive mesh 42 of the capture gel 40 overlies the bore 76.

The capture gel 40 can be affixed to the capture gel holder 66 by many methods including but not limited to ultrasonic welding, heat staking, and gluing. The polymer gel 44 component of the capture gel 40 can be cast onto the non-conductive mesh 42 before or after attaching the mesh to the holder 66. In one embodiment, the capture gel 40 placed on non-conductive mesh 42 before the mesh is attached to the holder 66.

An alternative electrophoresis cassette 86 that is used with the capture gel holder 66 is seen in FIGS. 19A-19C is used.

Referring to FIG. 19A, the electrophoresis cassette 86 has a base 88 and an evaporative cover 90. The evaporative cover 90 is transparent in one embodiment but is not limited to such.

The base 88 of the electrophoresis cassette 86 has a pair of electrode channels 92 and 94, as seen in FIG. 19B. The base 88 has a plurality of migration channels 96 which extend between the electrode channels 92 and 94. Within each migration channel 96, there is a pair of enlarged slots 98 adapted to receive a tooth 70 of the capture gel holder 66.

Each electrode channel 92 and 94 has an electrode 100 which extends the length of the respective electrode channel 92 and 94. In one embodiment, the electrodes 100 are each a pair of stainless steel pins 108, and a stainless steel strip 110 extends between the pins 108.

The evaporation cover 90 has a strip 102 which overlies the interface between the electrode channels 92 and 94 and the migration channel 96. Strip 102 restricts the flow of bubbles generated by buffer hydrolysis at the electrodes thereby facilitating their removal through vents 104 in the cover. The evaporative cover 90 has a plurality of vent holes 104 for overlying the electrodes 100. In addition, the evaporative cover 90 has holes 106 for receiving the teeth 70 of the capture gel holder 66 which overlies the enlarged slot 98 of the migration channel 96 of the base 88.

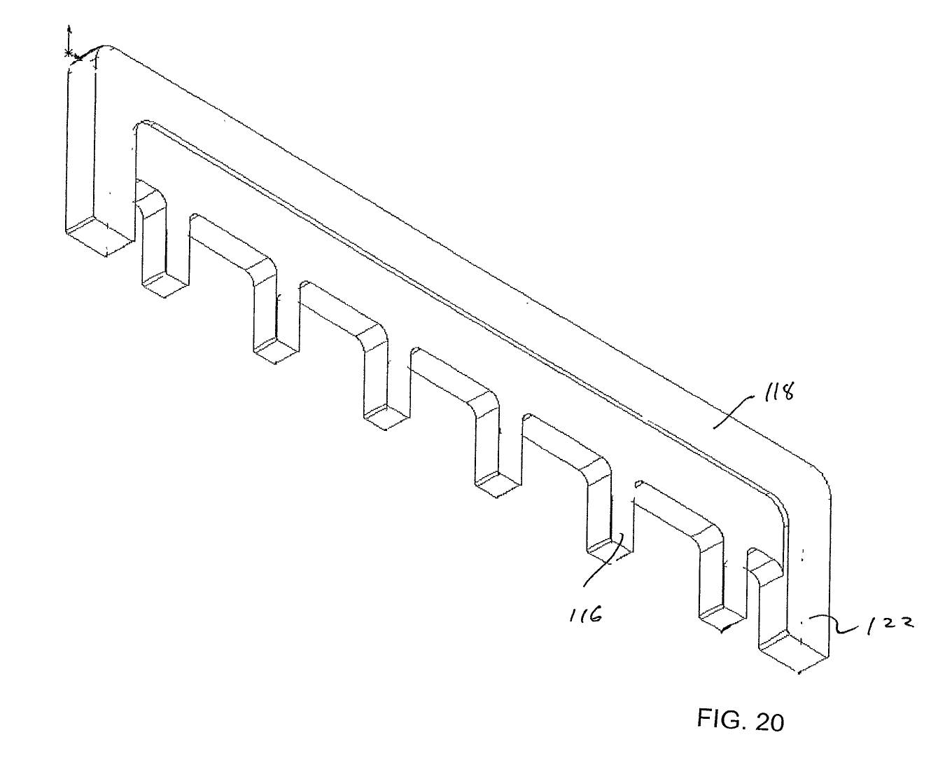

The evaporative cover 90 in addition has a plurality of generally square openings 114. The square openings 114 allow a plurality of teeth 116, as seen in FIG. 20, of a sample well-forming comb 118, to pass into the migration channel 96 as explained below.

Second Alternative Electrophoresis Cassette

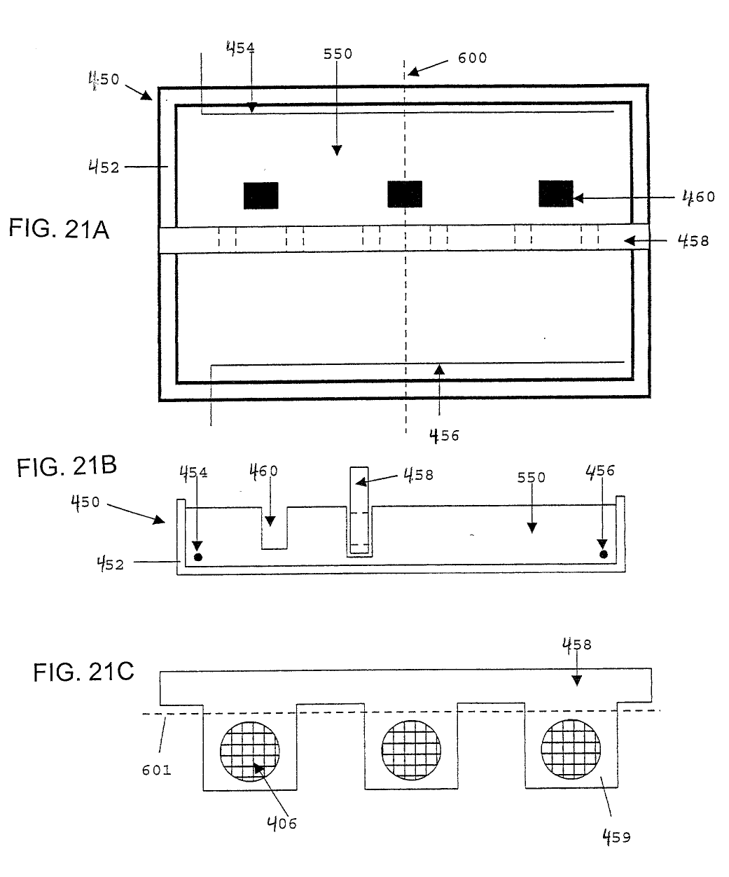

Referring to FIGS. 21A-21C, a second alternative electrophoretic cassettes 150 for use in the method of electrophoretically introducing a sample into a reinforced thin gel (or a thin gel) having capture probes covalently bound to the thin gel matrix is exemplified. The cassette 450 comprises a base 452, a first electrode 454, a second electrode 456, at least one holder 458 for holding reinforced thin gel 406, and an electrophoretic matrix 550. The electrophoretic matrix is buffer filled and substantially occupies the cassette base as a layer having a predetermined height. The cassette is provided with at least one well 460 in the electrophoretic matrix for receiving a liquid sample. The electrophoretic matrix can be formed from gel forming polymers such as for example agarose or starch. Alternatively, the electrophoretic matrix can be formed from a porous solid such as a polymeric sponge described in Harrington, et.al., U.S. Pat. No. 5,637,202, the disclosure of which is incorporated herein in its entirety by reference.

The reinforced thin gel is held in the proper orientation within the cassette by mean of the holder 458. In addition, the holder allows easy removal and transportation of the reinforced thin gel to a detection station for reading of the reinforced thin gel and detection of the target molecule when present in the sample. As shown in FIG. 21C, the holder, a gel capture holder, has at least one tooth 459 that extends down from a handle. The tooth has an aperture for receiving the reinforced thin gel. The holder is oriented so that the teeth of the holder extend down into the electrophoretic matrix when the holder is seated n the electrophoretic system. The holder positions the reinforced thin gel so that a target molecule in the sample in the well will migrate through the reinforced thin gel when a potential gradient is applied across the electrodes 454 and 456. In one embodiment of the electrophoretic system, the holder 458 fits into slots on the left and right sides of the base 452 to position the holder within the cassette.

The electrodes can be formed from many conductive materials including wire, carbon rods, metal strips, metal-plated fabric, conductive plastics, and the like. The conductive electrode materials can be formed as wires, thin strips, pins, rods, coatings deposited on the ends of the base 452, or conductive adhesive strips.

A preferred method for preparing the cassette of FIG. 21A is described below. First the electrodes and base are assembled. Then the holder 458 with attached reinforced thin gel 406 is placed into the base. The teeth of holder 458 are immersed in the matrix 550 up to the depth indicated by dashed line 601 in FIG. 21C. Then a comb with solid teeth is placed adjacent to, and parallel with the holder 158. An illustrative example of a comb 180 having a comb handle 182 and a tooth 184 is provided in FIG. 8B. An illustrative example of a holder 188 having a handle 182, a tooth 184, and an aperture 190 in the tooth for receiving a reinforced thin gel is provided in FIGS. 18A and 18B. It is preferred that the teeth of the comb for well formation are aligned with the teeth having apertures for receiving reinforced thin gel, so that in the finished cassette, the electric field drives the sample molecules in a straight path from the sample wells through the reinforced thin gels past the capture probes. Once the comb and holder are placed in the base 452, the base is filled with molten agarose in electrophoresis buffer. The filled cassette is allowed to cool and the agarose solidifies. After cooling, the sample well-forming comb is removed. A finished cassette is exemplified in FIG. 21A.

It will also be appreciated that while a specific embodiment of the present invention is the capture comb described herein, any holder or manifold capable of supporting the gel matrix is suitable for use in the methods described herein. A key characteristic of the holder is its ability to stable support the matrix (e.g. weld the mesh coated gel to the holder) in a manner suitable for electrophoresis. For example, many devices and manifolds commonly used for filtration or vacuum filtrations can be adapted for use as capture gel holders. For example, see the 96-well manifolds of the type used in the Multi screen™ product line (Millipore Corp. Bedford, Mass.).

While this invention has been particularly shown and described with references to preferred embodiments thereof, it will be understood by those skilled in the art that various changes in form and details may be made therein without departing from the scope of the invention encompassed by the appended claims.

### EXAMPLES

### Example 1

### Assay Procedure

Sample Processing

Draw 1 mL of sample material (e.g., platelet concentrate) and dispense into sample vials(s) containing 0.5 mL Sample Diluent. Cap the tubes and invert 3 to 5 times to mix. Centrifuge the tubes in a balanced microcentrifuge at 10,000+1,000×g for 1±0.25 minute. The control tubes are not centrifuged. Carefully remove the tubes from the rotor and remove the caps. Decant the supernatant by pouring into a BioHazard waste container, and briefly holding the lip of the tubes to a clean absorbent material. The tube should not be tapped or shaken.

Add 1 mL Rinse Buffer to each tube, resuspend by flicking the tube several times, and vortexing for 10 seconds, or until pellets are evenly resuspended. Recap and place in rack. Incubate the tubes at room temperature for 10-14 minutes. Centrifuge the tubes in a balanced microcentrifuge at 10,000±1,000×g for 1+0.25 minute. Once again, the control tubes are not centrifuged. Carefully remove the tubes from the rotor and remove the caps. Decant the supernatant by pouring into a BioHazard waste container, and briefly touch the lip of the tubes to a clean absorbent material. The tube should not be tapped or shaken.

Add 50±5 μl of Lysis Buffer into each sample tube. Tightly cap tubes, resuspend by flicking the tube several times, and vortexing for about 10 seconds, or until pellets are evenly resuspended. Obtain Negative Control (NC) and Positive Control (PC) tubes for each run. Place all tubes in the Lysis Unit (heater block), at 45° C. Let sit at 45° C. for about 5 minutes. Place control and sample tubes in heater 302 and heat to 124+4° C. Cool tubes to less than 50° C. Tubes now contain sample lysates appropriate for nucleic acid probe assays.

Hybridization

Before opening the tubes, shake down 3-4 times to bring most of the liquid to the bottom of the tubes. Add 25+3 μl of Probe Buffer to the samples and controls. Cap the tubes and mix by shaking down the tubes 3-4 times to bring most of the liquid to the bottom of the tubes. Place tubes in 45+2° C. heater for 10+1 minutes. Before opening the tubes, shake down 3-4 times to bring most of the liquid to the bottom of the tubes. Tubes now contain sample hybridization mixtures ready for electrophoresis. Insert comb into cassette prior to loading samples.

Electrophoresis

Load 25±3 μl from each tube into the sample well of a cassette 190 such as described with reference FIGS. 1-3, prewarmed on a 31±4° C. block. A new pipet tip should be used for each sample. Turn on the voltage to 40 volts, and run for 4 minutes, then reduce the voltage to 24 volts for another 16 minutes. Turn off the voltage, and move the membrane comb to the wash buffer wells, held at room temperature or in the warmed cassette, for ˜5 seconds. Move the comb to the electrophoretic wash slot, and turn on the voltage to 40 volts for 5±0.5 minutes. Turn off the voltage, and move the membrane comb to the Conditioning Buffer, hold at room temperature or in the warmed cassette, for 5-10 minutes.

Detection

Remove the comb from the cassette, and blot the sides of the comb with clean absorbent material held in the blotting fixture for 10 seconds. Place the comb into a reader tray, and place the reader tray into the reader. Add 25±31 μl room temperature substrate to each well and read.

Sample Diluent Buffer:

| NaH₂PO₄.monohydrate | 15 mM |
|---|---|
| Na₂HPO₄.heptahydrate | 15 mM |
| EDTA | 15 mM |
| Sodium Dodecyl Sulphate(SDS) | 0.30% |
| ProClin 5000 | 0.02% |
| pH | 7.0 +/− 0.1 |

Sample Rinse Buffer:

| | |
|---|---|
| NaH₂PO₄.monohydrate | 2.0 mM |
| Na₂HPO₄.heptahydrate | 4.0 mM |
| EDTA | 1.5 mM |
| Sodium Dodecyl Sulphate(SDS) | 0.03% |
| Sodium Chloride | 34 mM |
| Potassium Chloride | 1 mM |
| ProClin 5000 | 0.02% |
| Phenol Red | 0.001% |
| pH | 7.0 +/− 0.1 |

Lysis Buffer:

| | |
|---|---|
| NaH₂PO₄.monohydrate | 75 mM |
| Na₂HPO₄.heptahydrate | 75 mM |
| EDTA | 4.5 mM |
| Sodium Dodecyl Sulphate(SDS) | 7.50% |
| Dextran (188 kD) | 10% |
| Sulforhodamine B | 0.001% |
| ProClin 5000 | 0.02% |
| pH | 7.0 +/− 0.1 |

Probe Buffer:

| | |
|---|---|
| Tris Base | 25 mM |
| NaCl | 525 mM |
| Ficoll, 70 kDa | 5% |
| Xylene cyanole | 0.05% |
| Casein, Hammerstein Grade | 0.5 mg/mL |
| Sodium Azide | 0.04% |
| MgCl₂ | 0.5 mM |
| ZnCl₂ | 0.05 mM |

Reporter Probe alkaline phosphatase conjugated 2′-0-methyl ologonucleotides (see FIG. 9C for description of capture probes and reporter probes). 25 nM

pH 8.0+/−0.1

Wash Buffer:

| Tris Base | 90 mM |
|---|---|
| Boric Acid | 72 mM |
| Phosphoric Acid | 5.5 mM |
| SDS | 1% |
| Triton X-100 | 1% |
| ProClin5000 | 0.02% |
| BromPhenol Blue | 0.0005% |
| pH | 8.3 +/− 0.1 |

Conditioning Buffer:

| | |
|---|---|
| DEA | 100 mM |
| Magnesium Chloride | 0.1 mM |
| Zinc Chloride | 0.01 mM |
| Proclin 5000 | 0.02% |
| pH | 10.0 +/− 0.1 |

Alkaline Phosphatase Buffer:

| | |
|---|---|
| Tris Base | 25 mM |
| Sodium Chloride | 50 mM |
| Magnesium Chloride | 1 mM |
| Zinc Chloride | 0.1 mM |
| Sodium Azide | 0.04% |
| pH | 8.0 +/− 0.1 |

### Example 2

### Assay Using a Thin Gel Membrane.

In this example, cassettes similar to those of FIG. 19 were used with the following exceptions: 1) carbon rod electrodes were used in place of metal ones; 2) individual mesh reinforced thin gel membranes were used instead of using a multi-toothed capture gel holder. The individual thin gel membranes were manipulated with forceps.

Part I. Preparation of Thin Gel Membrane for Capture of Target Nucleic Acid

A. Preparation of Mesh

Polyester mesh with a thickness of 100 microns can be obtained from Sefar America, Inc., Briancliff Manor, NJ. The mesh is cut into rectangles (0.48 inch×0.52 inch) and washed by gentle shaking in ethanol (one wash of 5 minutes), deionized water (three washes of 5 minutes), and aqueous nonionic detergent (0.1% Tween 20, one wash for 5 minutes.) The mesh is air dried on clean filter paper.

B. Preparation of Gel Matrix Solution

Mix the following in a 1 ml tube: 337 microliters deionized water; 62 microliters 40% aqueous acrylamide solution 29:1 ratio of monomer to bisacrylamide (BioRad Laboratories, Richmond, Calif.); 50 microliters TrisBP buffer (TrisBP buffer is 4 parts of 900 mM Tris borate buffer (TrisBp) pH 8.3 mixed with 1 part 900 mM Tris phosphate buffer pH 8.3); 50 microliters 500 micromolar oligonucleotide capture probe with 5′ acrylamide modification, 5′-acrylamide-AGGCCCGGGAACGTATTCAC-3′ (SEQ ID NO: 18) (Acrydite™ modification, Mosaic Technologies, Boston, Mass.); 1 micro liter 20% TWEEN 20; and 3 microliters 10% TEMED (5 microliters in 45 microliters H2O)

C. Polymerization to form the thin gel membrane

Sandwich mesh rectangles between clean silanized glass plates. Stagger the plates slightly at one end so that on one end of the sandwich, the mesh lying against the bottom plate is exposed. Add 4 microliters 10% ammonium persulfate (50 mg in 500 microliters water) to the capture mix prepared in part B (above), mix, and spot the mixture onto exposed mesh, allowing the mixture to wick into the mesh between the glass plates. When the mesh is saturated, slide the top plate so that the mesh is completely sandwiched between the two plates. Allow polymerization for one hour at room temperature. Separate glass plates with a razor blade and trim excess polyacrylamide gel from the rectangular thin gel membranes. The membranes are washed by soaking them in a large volume of electrophoresis buffer (i.e., 0.1×TrisBP buffer).

Part II. Cassette Assembly

The cassette base used for this example was similar to that shown in FIGS. 19A-19C. Other cassette components include: one sample well-forming comb for forming sample wells in channel 301; and two carbon rod electrodes, wrapped with platinum wire (platinum wire is present for connection of carbon rods to the power supply). These cassette components are soaked in a solution of 1% hydrochloric acid and 1 millimolar (mM) EDTA (ethylenediaminetetraacetic acid) for at least 30 minutes to remove residual alkaline phosphatase activity. The components are then washed thoroughly with water and air dried on a clean surface.

Another set of cassette components is cleaned and dried including: a cassette base as shown in FIG. 19; two carbon rod electrodes, wrapped with platinum wire (platinum wire is present for connection of carbon rods to the power supply); and one sample well-forming comb (as shown in FIG. 20) for forming wells to hold the thin membrane within the cassette slots 98 during capture and washing. This second set will be used for electrophoretic washing of the thin gel membrane after target capture.

A molten solution of 1% agarose in 0.1×TrisBP is prepared in a 250 ml glass Ehrlenmeyer flask as follows. Mix: 100 ml 0.1×TrisBP buffer and 1 g agarose powder (Sigma A9539 General use Molecular Biology Grade). Stopper top of flask loosely with clean lab tissues (Kimwipes) and heat to boiling in a commercial microwave oven. Cover flask with aluminum foil and equilibrate to 60° C. in a water bath.

Assembly of the Sample Electrophoresis Cassette

Place a carbon electrode into each of the electrode channels (92 and 94 in FIG. 21) of the sample cassette. Pour molten agarose gel solution into the cassette, taking care to allow enough room so that the base is not overfilled when the comb is inserted. Remove any trapped air bubbles with a pipette tip or pasteur pipette. Adjust the position of the carbon electrodes against the outside edge within the electrode channels 92 and 94, to ensure that they are laterally symmetrical with respect to the sample electrophoresis channels.

Insert the sample well-forming comb 118 into the cassette base at the position of the slot 124 making sure that the teeth are centered in the channels, and the comb is not too close to the membrane slot.

Insert one thin gel membrane into each slot 98, making sure that the membrane is standing against the side of the sample well, and that the membrane is centered relative to the channel. Allow molten agarose gel to set for 30 minutes

Assembly of Washing Cassette (If a Separate Washing Cassette is Used)

Place the two carbon electrodes into the base as described above, and fill base with molten agarose gel solution. Insert membrane slot-forming comb as shown in FIG. 20 into slot 98. Check for any air bubbles, and reposition electrodes as described above. Allow the molten agarose gel to set for 30 minutes.

Part III. Cassette Operation

Sample Electrophoresis for Capture

Remove sample comb from the sample electrophoresis cassette, and fill the wells with electrophoresis buffer (0.1×TrisBP). Load 20 micro liter (ul) samples into the buffer-filled sample wells. Connect the platinum leads from the electrodes to a DC power supply and apply constant voltage of 50 V for 10 minutes. Temperature will increase during run, but should not be allowed to increase above 35 degrees centigrade (C). If necessary, the cassette can be placed on a surface cooled by a recirculating water bath to prevent overheating.

Electrophoretic Washing of Thin Gel Membranes

Pull well-forming comb from wash cassette and fill slots with electrophoresis buffer. Pull membranes out of the sample electrophoresis cassette slots 302 using forceps, and transfer each membrane to a buffer filled slot within the wash cassette. Removal of the membranes from the sample electrophoresis cassette is facilitated by rocking the membrane back and forth before pulling upward.

Connect electrodes to DC power supply and apply constant voltage of 50 V for 5 minutes taking care not to allow temperature of cassette to exceed 35 C.

### Example 3

### Rapid Format for Detection of Bacteria Using Non-Amplified Nucleic Acid Probes with Immobilized Capture Chemistry

A rapid, non-amplified diagnostic assay format based on the moderate copy-number 4.5S RNA from the bacterial signal recognition particle (SRP) is described herein. The SRP is a ribonucleoprotein complex which is present in Escherichia coli in 400-1000 copies per cell (depending on growth stage). (Batey et. al., Science 287:1232-1239; (2000); Jensen & Pedersen., J. Bacteriol. 176:7148-7154 (1994)). The 4.5S RNA or its homologue in bacteria varies considerably in size (˜79 nucleotides (nt) in Mycoplasma pneumoniae, 315 nt in Bacillus brevis) and in sequence. However, there is a conserved region of 22 nucleotides which is targeted for probe development.

Bacterial lysis is accomplished with heat and detergent, followed by rapid hybridization to a solution phase reporter probe conjugated to alkaline phosphatase (RP). The hybridization mixture is loaded into the well of a gel cassette, and subjected to rapid electrophoresis. The target RNA:RP complex is captured during electrophoresis by hybridization to capture probes (CP) which are immobilized with Acrydite™ chemistry in a polyacrylamide gel on a thin membrane. (Kenney et. al., Biotechniques 25:516-521 (1998); Rehman et. al. Nucleic Acids Research 27:649-655 (1999)).

Membranes are washed and exposed to chemiluminescent substrate for detection. The whole assay, including sample processing, can be conducted in about 60 minutes, with the following Signal-to-Noise values for E. coli:

| cfu/ml | mean +/− s.e.(n) |
|---|---|
| 1 × 105 | 3.9 +/− 0.4 (6) |
| 1 × 106 | 34 +/− 4 (6) |
| 1 × 107 | 309 +/− 60 (6) |

These data produce a power curve (log:log) fit of y=7.10⁻⁰⁵*(x0 9497), with R²=1.0. This assay format provides sensitivity for bacterial detection, with simple hybridization chemistry, standard chemiluminescent detection, and rapid time to first result.

### Example 4

### Rapid Assay for Bacterial Contamination

Bacteria was spiked into a platelet concentrate and enriched by centrifugation. The sample is lysised in the presence of sodium dodecyl sulphate at high temperature. The sample is hybridized with a high concentration of alkaline phosphatase-conjugated reporter probes. The selection of probes was based on published sequence information. (See, e.g., http://psyche.uthct.edu/dbs/srpdb/srpdb.html)

Acrydite™ attachment chemistry (U.S. Pat. No. 5,932,711) permits immobilization of capture probes at high concentration within a polyacrylamide gel matrix. Capture probe layers were supported on a thin membrane and inserted into an agarose-filled electrophoresis cassette 190, and subjected to electrophoresis of target molecules through the probe layer for rapid capture of targets and removal of excess unbound reporter probe.

The capture gel holder 220, capture membrane comb, was removed from the electrophoresis cassette 90, and inserted into a tray, the detection tray 78 of FIGS. 15A and 15B, designed to fit in a standard 96-well micro liter plate footprint. Chemiluminescent substrate was pipetted into these horizontal wells and light output was read in the luminometer over several minutes.

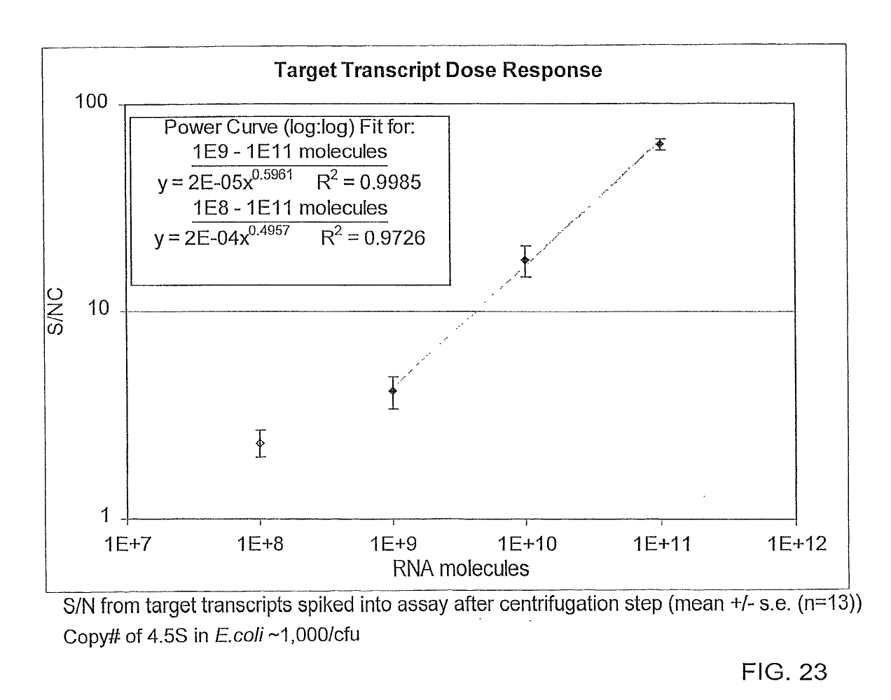

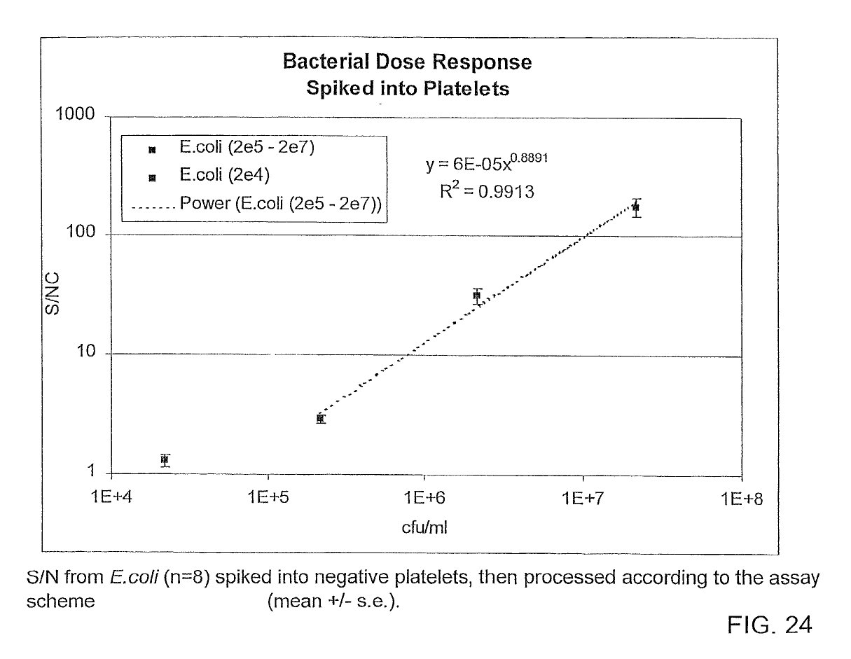

Chemiluminescence data are read and compared with the signal from a negative control matrix, consisting of BSA and nonspecific RNA, to calculate a Signal-To-Noise ratio (S/N). FIG. 23 shows a dose response for in vitro target transcripts spiked into the assay after the centrifugation step. FIG. 24 shows a similar dose response, but using E. coli spiked into sterile platelet concentrates.

Table 1 shows a list of the bacterial species which have been detected with the current probe set using the thin gel described herein.

TABLE 1
Bacteria Detected with Probe-Set Targeted to 4.5S RNA

| Genus | Species |
|---|---|
| Bacillus | cereus |
| Enterobacter | cloacae |
| Escherichia | coli |
| Klebsiella | pneumoniae |
| Pseudomonas | aeruginosa |
| Serratia | marcescens |
| Staphylococcus | aureus |
| Staphylococcus | epidermidis |
| Staphylococcus | warneri |
| Streptococcus | agalactiae |
| Streptococcus | pyogenes |

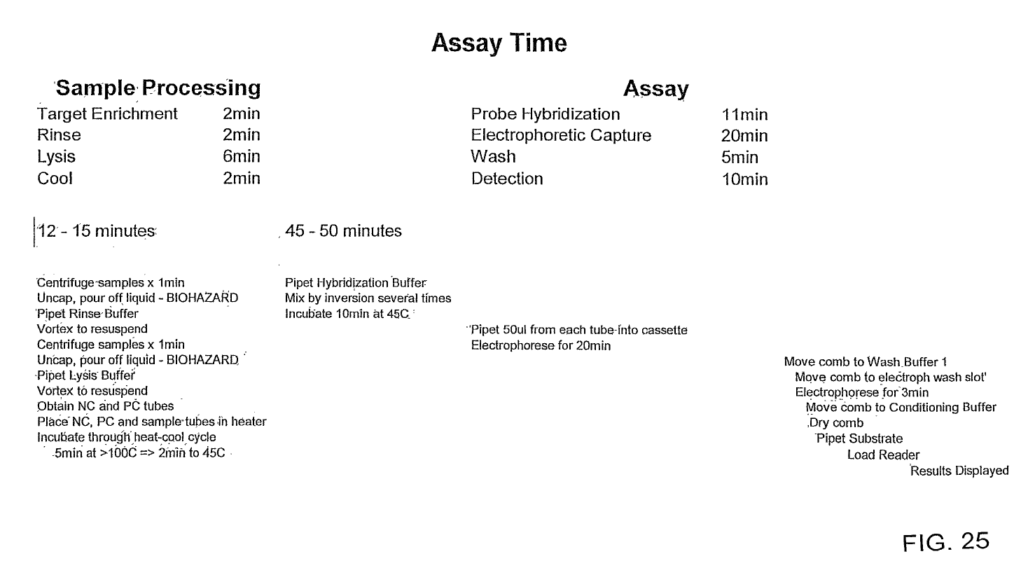

FIG. 25 depicts the time estimates for running up to 8 samples. (4 samples plus a negative and positive control per cassette, 2 cassettes run in parallel.)

### Example 5

### Formation of an Array

Individual rectangles are each embedded in a matrix forming gel solution having a unique probe. The rectangles, each provided with a specific probe or known probe set, are arranged in a known pattern between two clean silanized glass plates that are separated by spacers and sufficient acrylamide gel matrix is wicked in to form a thin gel surrounding each rectangle, joining the rectangles into a sheet.

### Example 6

### Blot Electrophoresis Procedure for Rapid Hybridization Analysis of Bacterial Colonies

Grow bacteria on petri plates. Blot onto nylon membrane coated with agarose. Lyse bacteria. Form sandwich of nylon membrane and silk screen mesh with each outer surface of the sandwich covered with Saran Wrap and with a plate spacer between the nylon membrane and the silk screen mesh. Wick thin gel matrix forming solution between the silk screen and nylon meshes, and into any open space in the silk screen and nylon mesh. Allow solution to gel.

Place the gel sandwich into a cassette filled with unsolidified agarose and allow the agarose to solidify. Apply a current to the electrodes thereby bringing any analyte in the agarose on the nylon membrane proximal to the ligand bound to the thin gel matrix.

Introduce a label capable of binding to an analyte-ligand complex and allow it to bind. Detect the presence or absence of the label by removing the thin gel matrix from the cassette and moving it to the reader for the label.

### Example 7

### Detection of E. coli Contamination in Platelet Concentration

A sample was prepared for detection of E. coli contamination in platelet concentrates. In this example, log phase E. coli were added to a platelet sample to provide a simulation of a contaminated platelet sample. The nucleic acid probes, the probes 46 on the capture gel 40, used are directed toward detection of a target, E. coli 4.5S RNA, also known as the signal recognition particle RNA. The alkaline phosphatase conjugated detection probe hybridizes directly to the 4.5S RNA target. The adaptor probe (block 154 of FIG. 10) also hybridizes directly to the 4.5S RNA target at a location distinct from that occupied by the detection probe. One portion of the adaptor probe is not complementary to the target and forms a single stranded tail when hybridized to the target 4.5S RNA. This single stranded region of the adaptor is complementary to the capture probe which is immobilized in the reinforced thin gel. (See for example, PCT/U.S.00/08529, the teachings of which are incorporated herein by reference.)

The concentration and lysing of the sample is discussed referring to block 150 of FIG. 10. One (1.0) milliliter (ml) samples of platelets were drawn and dispensed into labeled tubes (polypropylene screw-cap storage vials for cryogenic storage, approximately 2 ml volume, Nalge). Log phase E. coli bacteria in TE buffer, (TE is 10 mM Tris HCl pH 8.3, 1 mM EDTA) were spiked into platelet samples. “Negative” sample receives TE buffer only. Other “positive” samples received, 5×1 colony forming units (cfu), 5×10⁶ cfu, and 5×10⁷ cfu. All the samples were spun for 60 seconds at 14,000 rpm in microcentrifuge (e.g., Eppendorf).

The supernatant were poured off into biohazard waste and the lip of tube was blotted on clean paper towel. One hundred (100) ul lysis buffer (lysis buffer is 100 mM sodium phosphate buffer pH 7.0, 3 mM EDTA, 5% SDS, 0.001% phenol red) is delivered to each tube. The pellet is resuspend by vortex mixing or by pipetting up and down with a micropipette.

Each tube was capped tightly and placed into a pre-warmed 135° C. heating block for 5 minutes as illustrated by block 152 in FIG. 10. After heating, the tubes are moved to a room temperature rack for at least 90 seconds. In one embodiment, the tubes are placed in an incubation unit 304, such as shown in FIG. 14, which both heats and cools. Twenty (20) μl of 5×DHB+AP is added to ea. tube with Eppendorf repeater (5×DHB+AP is 525 mM NaCl, 0.5 mM MgCl₂, 0.05 mM ZnCl₂, 125 mM Tris HCl pH 8.3, 5% Ficoll 400, 0.05% wt/vol xylene cyanole, 125 nanomolar (nM) alkaline phosphate conjugated oligondeoxynucleotide probe (3-CTTCC GTCTA CTGCG CACAC GG-alkaline phosphatase-5′) (SEQ ID NO: 19), 2.5 mM aurin tricarboxylic acid, 125 nM adaptor oligonucleotide probe (3′-TCCGG GCCCT TGCAT AAGTG AGTCC AGGCC TTCCT TCGTC G-5′) (SEQ ID NO: 20), 0.05% sodium azide). Each tube is manually shaken several times. The tubes are incubated at 52° C. for 10 minutes to hybridize the reporter probe and adaptor probe to the 4.5S RNA from the bacteria. Twenty (20) ul of the hybridization mixture is loaded into the sample wells 174 in cassette 86 of FIG. 19B.

The electrodes 100 are connected to a DC power supply and apply constant voltage of 50V for 10 minutes. Temperature will increase during the run, but should not be allowed to increase above 35 degrees centigrade (C). If necessary, the cassette 86 can be placed on a surface cooled by a recirculating water bath to prevent overheating.

Referring to block 158 of FIG. 10, after a set period of time of migration of the sample towards the capture gel 40 in the capture gel holder 66, the power is turned off. The capture gel holder 66 is moved from the enlarged segment 98 in the flow channel 96 to the other enlarged segment 98 in the same electrophoresis cassette 86. The removal of the membranes from the sample electrophoresis cassette is facilitated by rocking the membrane back and forth before pulling upward.

The capture gel holder is moved from the first set of enlarged slots 98 of the electrophoresis cassette 86 and transferred to the second set of enlarged slots, the wash segment, 98 of the electrophoresis cassette 86. The teeth 70 of the capture gel holder 66 are placed in the buffer-filled slot created by the enlarged slot 98. The electrodes 100 are connected to a DC power supply and a constant voltage of 50 V for 5 minutes is applied. Once again, care is taken not to allow the temperature of the cassette to exceed 35° C.

As indicated above, when discussing the method in relation to FIG. 10, the current is in the same direction during the second or wash period. By moving the capture gel holder 66 to the second enlarged segment 98, the prorogation of the sample is away from the capture gel 40 of the capture gel holder 66 and thus any sample on the capture gel holder 66 that will not be captured will pass through and no new sample will come in contact with the capture gel 40.

After the above steps, the detection of captured nucleic acid target-alkaline phosphatase conjugated probe complex on thin gel membranes, the capture gel 40 of the capture gel holder 66, is accomplished. The capture gel holder 66 is removed from the wash or second set of slots 98 of the electrophoresis cassette. The teeth 70 with the capture gel 40 are placed in Tropix reagent (CDP-star with Emerald II, Tropix catalog MS100RY). The capture gel 40 is soaked in the solution and incubated at room temperature with gentle rocking or shaking for 15 minutes, as illustrated in FIG. 8, by reference numeral 170.

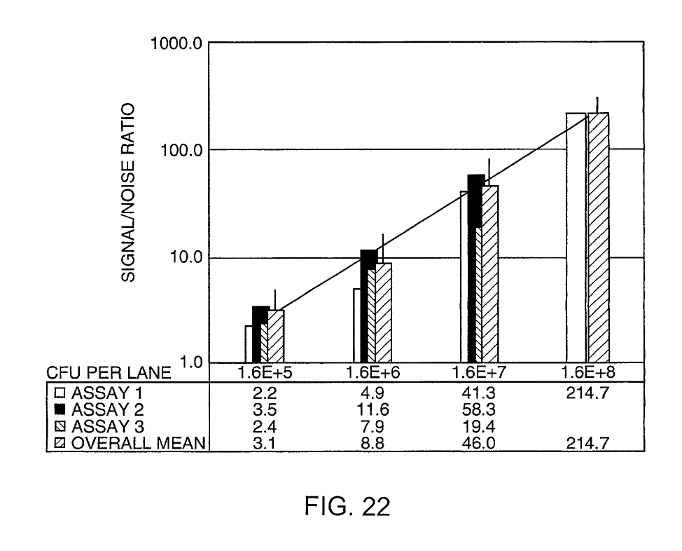

The capture gel holder 66 is then placed in a detection tray 178, as shown in FIGS. 15A & 15B. The capture gel holder 66 and the detection tray 178 are placed in a microplate luminometer 172 such as a Walla Victor 2, E.G.&G Walla, Turku, Finland. Relative light units (RAU) are read and both the RAU and signal to noise ratio=(Test sample membrane minus empty well)/(Negative control sample membrane minus empty well) are reported FIG. 22 illustrates the results of three different experiments (assays 1 through 3); results are displayed as a signal to noise ratio (S/N). The overall mean S/N ratio is also displayed along with its standard deviation. The samples tested comprise human platelet concentrates spiked with the indicated number of log-phase E. coli bacteria (expressed as the number of colony forming units (cfu) loaded per lane). The expression “1.6E+5” is an abbreviation for the value “1.6×10⁵”.

What is claimed is:

1. An apparatus for capturing an analyte comprising:
an electrophoresis cassette including:
a base having a pair of electrode channels, a barrier interposed between the electrode channels, the barrier having at least one migration channel extending between the electrode channels, an enlarged slot bounded and opening into the migration channel;
a first electrode extending in the first electrode channel; and
a second electrode extending in the second electrode channel; and
a capture gel holder receivable in the enlarged slot, the capture gel holder having an opening aligned with the migration channel.

2. The apparatus of claim 1 wherein the barrier has a second enlarged slot bounded and opening into the migration channel for receiving the capture gel holder.

3. The apparatus of claim 2 further comprising an evaporation cover for overlying the electrophoresis cassette, the evaporation cover having at least one opening for the capture gel holder and at least one opening for venting of gas.

4. The apparatus of claim 3 wherein the electrophoresis cassette includes at least one wash well.

5. The apparatus of claim 3 wherein at least one of the electrodes has a pair of terminals that extend through the evaporation cover and are flush with the top of the evaporation cover.

6. The apparatus of claim 3 wherein the capture gel holder has a plurality of teeth, each tooth having an opening for receiving non-conductive polymeric mesh and the teeth having a polarity device so that the teeth fit in the enlarged slot of the electrophoresis cassette in a certain manner.

7. The apparatus of claim 4 wherein the gel capture holder has a plurality of teeth, each tooth having an opening for receiving non-conductive polymeric mesh and the teeth having a polarity device so that the teeth fit through the at least one opening of the evaporation cover.

8. An apparatus for capturing an analyte comprising:
an electrophoresis cassette including:
a base having a pair of electrode channels, a barrier interposed between the electrode channels, the barrier having at least one migration channel extending between the electrode channels, an enlarged slot bounded and opening into the migration channel;
a first electrode extending in the first electrode channel; and
a second electrode extending in the second electrode channel;
a capture gel holder receivable in the enlarged slot, the capture gel holder having an opening aligned with the migration channel;
a thin gel carried in the opening of the capture gel holder, the thin gel having a gel matrix and a ligand covalently bound to the gel matrix.

9. The apparatus of claim 8 wherein the thin gel further comprising a non-conductive polymeric mesh for linking with the gel matrix.

10. The apparatus of claim 9 wherein the barrier has a second enlarged slot bounded and opening into the migration channel for receiving the capture gel holder.

11. The apparatus of claim 10 further comprising an evaporation cover for overlying the electrophoresis cassette, the evaporation cover having at least one opening for the capture gel holder and at least one opening for venting of gas.

12. The apparatus of claim 11 wherein the electrophoresis cassette includes at least one wash well.

13. The apparatus of claim 11 wherein at least one of the electrodes has a pair of terminals that extend through the evaporation cover and are flush with the top of the evaporation cover.

14. The apparatus of claim 11 wherein the capture gel holder has a plurality of teeth, each tooth having an opening for receiving non-conductive polymeric mesh and the teeth having a polarity device so that the teeth fit in the enlarged slot of the electrophoresis cassette in a certain manner.

15. The apparatus of claim 14 wherein the capture gel holder further comprises a detection surface.

16. The apparatus of claim 11 wherein the gel capture holder has a plurality of teeth, each tooth having an opening for receiving non-conductive polymeric mesh and the teeth having a polarity device so that the teeth fit through the at least one opening of the evaporation cover.

17. A capture gel holder comprising:
a handle;
a plurality of teeth projecting from the handle, at least one of the teeth having a bore through the tooth; and
a gel matrix and a ligand covalently bound to the gel matrix overlying the bore.

18. The capture gel holder of claim 17 wherein at least one tooth having a keyed shaped therein adapted to fit in the electrophoresis cassette in only in a specific direction.

19. The capture gel holder of claim 18 wherein each of the teeth has a recessed central region around the bore and a flange on the bore wherein the recessed region and the flange are to facilitate the release of gas.

20. The capture gel holder of claim 19 further comprising a non-conductive polymeric material overlying each bore of the teeth and for supporting the gel matrix and ligand.

21. The capture gel holder of claim 20 wherein the keyed shape includes each tooth having a curved edge and a flat edge adapted to be fit in the electrophoresis cassette only in a specific direction.

22. The capture gel holder of claim 21 wherein the keyed shape includes one tooth having a protrusion adapted to pass through only a specific slot in an evaporative cover of an electrophoresis cassette.

23. The apparatus of claim 21 wherein the capture gel holder further comprises a detection surface.

24. The apparatus of claim 20 wherein the keyed shape includes one tooth having a protrusion adapted to pass through only a specific slot in an evaporative cover of an electrophoresis cassette.

25. A thin gel for capturing an analyte by electrophoresis comprising:
a gel matrix and a ligand covalently bound to the gel matrix.

26. The thin gel of claim 25 wherein the gel matrix has a tensile strength sufficient to allow removal from the electrophoresis device.

27. The thin gel of claim 25 further comprising a non-conductive polymeric material.

28. The thin gel of claim 27 wherein the non-conductive polymeric material is selected from the group consisting of a mesh, a mat, a woven fabric, and a felt.

29. The thin gel of claim 27 wherein the non-conductive polymeric material is a polymer capable of cross-linking the gel matrix.

30. An apparatus for capturing an analyte, the apparatus comprising:
a gel matrix and a ligand covalently bound to the gel matrix.

31. The apparatus of claim 30 further comprising a non-conductive polymeric material for supporting the gel matrix and ligand.

32. The apparatus of claim 31 further comprising a capture gel holder having a plurality of openings for receiving the non-conductive polymeric material.

33. The apparatus of claim 32 further comprising an electrophoresis cassette for receiving a gel for electrophoresis and capturing an analyte, the electrophoresis cassette includes
a base having a pair of electrode channels, and a barrier interposed between the electrode channels, the barrier having at least one migration channel extending between the electrode channels; and an enlarged slot bounded and opening into the migration channel for receiving the capture gel holder;
a first electrode extending in the first electrode channel; and
a second electrode extending in the second electrode channel.

34. The apparatus of claim 33 wherein the barrier has a second enlarged slot bounded and opening into the migration channel for receiving the capture gel holder.

35. A method of capturing a target molecule contained in a sample, the method comprising the steps of:
providing a non-conductive polymeric material having a gel matrix comprising a covalently bound ligand specific for the target molecule; and
passing the sample through the non-conductive polymeric material having the gel matrix such that the target molecule is captured by the capture probe of the gel matrix and the remainder of the sample passes through the non-conductive polymeric material.

36. The method of claim 35 wherein the target molecule is detectably labeled prior to passing the sample through the gel matrix.

37. The method of capturing of the target molecule of claim 29 further comprising the steps of:
providing an electrophoresis cassette having a migration channel extending between a pair of electrode channels each having an electrode;
providing an electrophoretic matrix in the electrophoresis cassette and forming a sample-well to receive the sample within the migration channel;
inserting the non-conductive polymeric material having the gel matrix comprising the covalently bound ligand which is carried by a capture gel holder into the migration channel by placing the capture gel holder into an enlarged slot which bounds and opens into the migration channel;
inserting the sample with the target molecule in the sample well;
passing a voltage in the electrophoresis cassette to cause the sample to migrate in the channel from the sample well towards the non-conductive polymeric material.

38. The method of claim 37 further comprising the steps of:
removing the capture gel holder from the electrophoresis cassette; and
placing the capture gel holder in a reader to detect a probe associated with the analyte.

39. The method of claim 38 wherein the electrophoresis cassette has a second enlarged slot for a respective migration channel for receiving the capture gel holder.

40. The method of claim 37 further comprising the steps of:
removing the capture gel holder from the electrophoresis cassette; and
subjecting the capture gel holder to conditions sufficient to break the bond between the capture probe and the target molecule.

41. The method of claim 40 wherein the electrophoresis cassette has a second enlarged slot for a respective migration channel for receiving the capture gel holder.

42. A method of detecting a target molecule comprising the steps of:
providing a capture gel holder having a non-conductive polymeric material having a gel matrix comprising a covalently bound ligand;
providing an electrophoresis cassette having a migration channel extending between a pair of electrodes and a sample well to receive the sample within the migration channel and a pair of enlarged slots bounding and opening into the migration channel;
inserting the sample with the target molecule in the sample well;
inserting the capture gel holder into one of the pair of enlarged slots in the migration channel;
passing a voltage in the electrophoresis cassette to cause the sample to migrate in the migration channel from the sample well towards the non-conductive polymeric material;
removing the capture gel holder from the electrophoresis cassette; and
placing the capture gel holder in a reader to detect a probe associated with the analyte.

43. The method of claim 42 further comprising the steps of:
preparing the sample including having a reporter probe to adhere to the target molecule;
stopping the voltage in the electrophoresis cassette;
moving the capture gel holder to a wash station inserting the capture gel holder into the other enlarged slot in the migration channel; and
passing a voltage through the electrophoretic matrix in the electrophoresis cassette to cause the sample to migrate in the channel from the sample well away from the capture gel holder and the non-conductive polymeric material.

44. A method for performing an analyte ligand binding assay, said method comprising the steps of:
a.) providing an electrophoresis cassette having
a base unit having a pair of electrode channels, a barrier interposed between the electrode channels, the barrier having at least one migration channel extending between the electrode channels; and an enlarged slot bounded and opening into the migration channel for receiving the capture gel holder;
a first electrode extending in the first electrode channel;
a second electrode extending in the second electrode channel;
a sample-well forming comb removable seated in the migration channel; and
a thin gel having a gel matrix and a ligand covalently bound to the gel matrix and placed in the migration channel;
b.) filling the apparatus with a gel for electrophoresis;
c.) allowing the gel to solidify;
d.) removing the comb to thereby create a sample well;
e.) placing a sample in the sample well;
f.) providing an electromotive force; and
g.) moving the sample through said thin gel with said electromotive force.

45. The method of claim 44 further including the step of providing a detection probe in said sample well.

46. The method of claim 44 further including the steps of: g.) removing said thin gel from said apparatus and h.) detecting the presence or absence of said detection probe.

47. The method of claim 46 further including the step of washing said thin gel between steps g.) and h.).

48. An apparatus for capturing a target molecular comprising:
a housing having:
a base having a first electrode channel, a second electrode channel, a barrier interposed between the first electrode channels and the second electrode channel, the barrier having at least one migration channel extending between the first electrode channels and the second electrode channel, an enlarged slot bounded and opening into a the migration channel;
a slot, the capture gel holder having an opening aligned with the migration channel; and
a gel carried in the opening of the capture gel holder, the thin gel having a gel matrix and a ligand covalently bound to the gel matrix.

49. The apparatus of claim 48, wherein said apparatus further comprises a first electrode extending in the first electrode channel and a second electrode extending in the second electrode channel.

50. The apparatus of claim 48 wherein the gel further comprises a non-conductive polymeric mesh for linking with the gel matrix.

51. The apparatus of claim 50 wherein the barrier has a second enlarged slot bounded by and opening into the migration channel for receiving the capture gel holder.

52. The apparatus of claim 51 further comprising an evaporation cover for overlying the electrophoresis cassette, the evaporation cover having at least one opening for the capture gel holder and at least one opening for venting of gas.

53. The apparatus of claim 52 wherein the electrophoresis cassette includes at least one wash well.

54. The apparatus of claim 52 wherein at least one of the electrodes has a pair of terminals that extend through the evaporation cover and are flush with the top of the evaporation cover.

55. The apparatus of claim 52 wherein the capture gel holder has a plurality of teeth, the teeth having a polarity device so that the teeth fit in the enlarged slot of the electrophoresis cassette in a certain manner.

56. A capture gel holder comprising:
a handle;
a plurality of teeth projecting from the handle, at least one of the teeth having a bore through the tooth; and
a gel matrix overlying the bore.

57. The capture gel holder of claim 56 wherein at least one tooth has a keyed shaped to permit placement therein adapted to fit in the electrophoresis cassette in only in a specific direction.

58. The capture gel holder of claim 57 wherein each of the teeth has a recessed central region around the bore and a flange on the bore wherein the recessed region and the flange are capable of facilitating release of gas.

59. The capture gel holder of claim 58 further comprising a non-conductive polymeric material overlying each bore of the teeth and for supporting the gel matrix and ligand.

60. The capture gel holder of claim 59 wherein the keyed shape includes each tooth having a curved edge and a flat edge adapted to be fit in the electrophoresis cassette only in a specific direction.

61. The capture gel holder of claim 60 wherein the keyed shape includes one tooth having a protrusion adapted to pass through only a specific slot in an evaporative cover of an electrophoresis cassette.

62. The apparatus of claim 60 wherein the capture gel holder further comprises a detection surface.

63. The apparatus of claim 59 wherein the keyed shape includes one tooth having a protrusion adapted to pass through only a specific slot in an evaporative cover of an electrophoresis cassette.

64. A gel for capturing an analyte by electrophoresis comprising:
a gel matrix and a ligand covalently bound to the gel matrix.

65. The gel of claim 64 wherein the gel matrix has a tensile strength sufficient to allow removal from the electrophoresis device.

66. The gel of claim 64 further comprising a non-conductive polymeric material.


SEQUENCE LISTING (20 sequences)

SEQ ID NO: 1 - DNA, 50 bp, Staphylococcus epidermis
catgaaccatgtcaggtcctgacggaagcagcattaagtggatcctcata

SEQ ID NO: 2 - DNA, 26 bp, Unknown
  staphylococcus
acatgtcaggtcctgacggaagcagc

SEQ ID NO: 3 - DNA, 50 bp, Staphylococcus aureus
catgaaccatgtcaggtcctgacggaagcagcattaagtggatcatcata

SEQ ID NO: 4 - DNA, 49 bp, Unknown
  staphylococcus
catgaaccatgtcaggtcctgacggaagcagcattaagtggatctcata

SEQ ID NO: 5 - DNA, 46 bp, Streptococcus pyogenes
cttgtgcgtgaagtgggtcaggggaggaatccagcagccctaagcc

SEQ ID NO: 6 - DNA, 46 bp, Streptococcus pneumoniae
gttctgcgtgaagcgggtcaggggaggaatccagcagccctaagcg

SEQ ID NO: 7 - DNA, 26 bp, Unknown
  streptococcus
atgggtcaggggaggaatccagcagc

SEQ ID NO: 8 - DNA, 46 bp, Streptococcus equi
cttgtgcgtgaagtgggtcaggggaggaatccagcagccataagcg

SEQ ID NO: 9 - DNA, 46 bp, Streptococcus mutans
gctttgcgtgaagcgggtcaggggaggaatccagcagccctaagcg

SEQ ID NO: 10 - DNA, 41 bp, Unknown
  streptococcus
ttgcgtgaaggggtcaggggaggaatccagcagcctaagcg

SEQ ID NO: 11 - DNA, 64 bp, Klebsiella pneumoniae
cgcaacgctactctgtttaccaggtcaggtccggaaggaagcagccagagcagacgacgtgtgt

SEQ ID NO: 12 - DNA, 64 bp, Unknown
  styphimur W
cgcaacgctactctgtttaccaggtcaggtccggaaggaagcagccaaggcagatgacgcgtgt

SEQ ID NO: 13 - DNA, 64 bp, Escherichia coli; identical to SEQ ID NO: 12

SEQ ID NO: 14 - DNA, 26 bp, Escherichia coli
acaggtcaggtccggaaggaagcagc

SEQ ID NO: 15 - DNA, 64 bp, Pseudomonas aeruginosa
cgcaacgattacccgtcaacctggtcaggtccggaaggaagcagccacagcgggaacatcgtgt

SEQ ID NO: 16 - DNA, 64 bp, Bacillus cereus
cgcaacgggacccgtgaaccttgtcaggtccggaaggaagcagcaataagcggtcttctcgtgt

SEQ ID NO: 17 - DNA, 44 bp, Artificial Sequence
  oligonucleotide probe
cgcaacgcgtaccggtcaggtccggaaggaagcagcagcggtgt

SEQ ID NO: 18 - DNA, 20 bp, Artificial Sequence
  oligonucleotide probe
aggcccgggaacgtattcac

SEQ ID NO: 19 - DNA, 22 bp, Artificial Sequence
  oligonucleotide probe
cttccgtctactgcgcacacgg

SEQ ID NO: 20 - DNA, 41 bp, Artificial Sequence
  oligonucleotide probe
tccgggcccttgcataagtgagtccaggccttccttcgtcg